(19) United States
(12) Patent Application Publication
(10) Pub. No.: US 2019/0209475 A1
(43) Pub. Date: Jul. 11, 2019

(54) FVIII Formulation

(71) Applicant: Novo Nordisk A/S, Bagsvaerd (DK)

(72) Inventors: Thomas Nylandsted Krogh, Jyllinge (DK); Michael Bech Jensen, Alleroed (DK); Heidi Westh Bagger, Koebenhavn S (DK)

(21) Appl. No.: 15/772,538

(22) PCT Filed: Nov. 3, 2016
(86) PCT No.: PCT/EP2016/076548

§ 371 Date: May 1, 2018

(30) Foreign Application Priority Data
Nov. 5, 2015 (EP) 15193099.7

(51) Int. Cl. A61K 9/19; A61K 38/37; A61K 9/00; A61K 47/26; A61K 47/18; A61P 7/04

(52) U.S. Cl. CPC A61K 9/19; A61K 38/37; A61P 7/04; A61K 47/26; A61K 47/18; A61K 9/0019

(57) ABSTRACT

The present invention relates to pharmaceutical formulations, in particular FVIII formulations. The present invention furthermore relates to methods for producing such compositions.

### TECHNICAL FIELD

The present invention relates to pharmaceutical formulations, in particular FVIII formulations.

### BACKGROUND

Haemophilia is an inherited bleeding disorder: Formation of the blood clot in the patients occurs normally but the clot is unstable due to a lack of secondary thrombin formation. The disease is treated by intravenous (iv) injection of coagulation factors such as e.g. factor FVII (FVII), Factor VIII (FVIII), or Factor IX (FIX) isolated from blood or produced recombinantly. The iv coagulation factor formulations are freeze dried formulations that are reconstituted in water, saline or buffer prior to use. Current haemophilia treatment recommendations are moving from traditional on-demand treatment towards prophylaxis, preferably using longer acting FVIII variants having a prolonged in vivo circulatory half-life.

Intravenous (iv) infusions with coagulation factors, in particular frequent iv infusions, are considered inconvenient, stressful, painful, and may even be traumatising for the patients and/or associated with risk of infection. Some haemophilia patients have poor venous access and many are young infants. Intravenous administration may furthermore be associated with low compliance.

Subcutaneous (sc) administration, on the other hand, is normally considered convenient and pain-free, or nearly pain-free. Sc administration of FVIII is furthermore thought to provide patients with a relatively high and constant trough FVIII level, e.g. in connection with daily administration. Sc administration of coagulation factors (e.g. FVIII) has, however, thus far not been feasible due to the (too) low bioavailability of the protein in connection with subcutaneous administration, as well as due to other types of obstacles related to the formulations.

FVIII iv formulations currently available are generally characterised by having a relatively high osmolality (about 400-600 mOsm/kg combined with a relative large reconstitution volume of about 4-5 mL), high salt contents, relatively low carbohydrate content (e.g. sugar/sucrose), relatively high injection volumes (4-5 mL), and relatively low drug concentration (50-750 IU/mL).

In contrast, a pharmaceutical formulation for sc administration should preferably have a relatively low injection volume. The injection volume of a sc formulation should be limited to 2 mL or less, preferably 1.5 mL or less and most preferably 1 mL or less than 1 mL.

The formulation for sc administration should preferably have a lower osmolality compared to the iv formulations, e.g. be isotonic, or close to isotonic. Hypertonic formulations (e.g. formulations comprising high salt contents) may cause injection site reactions and/or pain in connection with sc administration. Pain and local irritation associated with subcutaneous injection can be caused by too high or too low tonicity combined with relatively high injection volume. Currently available iv FVIII formulations are thus not suitable for subcutaneous administration.

### SUMMARY

The present invention relates to a pharmaceutical FVIII formulation, wherein said formulation comprises a FVIII molecule having an increased in vivo circulatory half-life (“long acting FVIII”), compared to wt FVIII, wherein said formulation is an aqueous, essentially isotonic formulation following reconstitution (into a volume relevant for sc administration), and wherein said formulation comprises 250-10,000 IU (/mL) of said FVIII molecule, 2-7 mg NaCl/mL, 3.4-34 mM CaCl₂ (0.5-5.0 mg CaCl₂.2H₂O/mL), 50-110 mg sucrose/mL, and optionally 0.5-15 mg methionine/mL.

The present invention furthermore relates to methods for producing such compositions as well as products produced by such methods as well as therapeutic use thereof.

### DESCRIPTION

The majority of recombinant FVIII products on the market (e.g. NovoEight®, Refacto®, Kogenate®, Advate®, etc.) are freeze dried products. These FVIII formulations have been designed for iv infusion with relative large reconstitution volumes and relative large injection volumes. The freeze dried iv FVIII products are usually reconstituted in 4-5 mL of either sterile water for injection (WFI) or aqueous saline/buffer solutions. After reconstitution, the resulting reconstituted FVII formulations/solutions have osmolalities of about 400-600 mOsm/kg. This can be described as slightly hypertonic to hypertonic iv solutions with no safely concerns in relation to iv infusion, where formulations are immediately diluted in the blood stream.

The concentration of drug in the reconstituted iv FVIII products is relatively low, about 50-750 IU/mL (roughly corresponding to 5-80 μg/mL) and relatively large volumes are therefore injected to provide the target doses.

Currently available FVIII formulations for iv infusion are in general not suitable for sc administration. This is primarily due to safety restrictions regarding injection volume and tonicity (osmolality). The volume limit per sc injection is normally about 1-2 mL, and preferably less than 1 mL. Formulations for sc administration should preferably be isotonic, or close to isotonic. A combination of large injection volume and hyper-tonicity is not regarded as a safe sc injectable. Since the osmolality is primarily controlled by the molal concentration of dissolved components (protein and excipients), it is not desirable to reduce the reconstitution volume to 1 mL for currently available recombinant iv FVIII products as this would result in a very hypertonic solution with an osmolality of about 1-1.5 Osm/kg (1000-1500 mOsm/kg). Furthermore, commercially available FVIII products normally contain Polysorbate 80/“Tween® 80”—a surfactant that may increase the risk for injection site reactions/irritations upon sc administration.

The bioavailability of FVIII in connection with sc administration is very low—but the inventors have recently discovered that the sc bioavailability of long acting FVIII molecules (e.g. certain FVIII fusion proteins, conjugated FVIII, etc.) is surprisingly high compared to sc administration of wt FVIII. The inventors have herein furthermore discovered that a long acting FVIII molecule can be formulated, freeze dried and subsequently reconstituted in a volume of about 1 mL (or less, such as e.g. 0.8 mL, 0.5 mL, or 0.3 mL) while having an acceptable FVIII concentration and an osmolality of about 350-500 mOsm/kg (close to isotonic or slightly hypertonic). However, the inventors also discovered that in connection with some sc FVIII formulations, the FVIII light chain had a tendency to become oxidised. Other obstacles related to the provision of a sc FVIII formulation was to provide a nice appearing freeze dried formulation with stable long acting FVIII, said formulation having low osmolality (isotonic or close to isotonic) upon reconstitution with 1 mL or less.

The excipients in freeze dried formulations should form a matrix providing the requisite stabilization of the formulated protein. Some excipients tend to form crystals during freeze drying. The self-interacting nature of a crystal may reduce the stabilizing and cryo-protecting properties of crystallized excipients in a formulation. Melting during freeze drying, which may result in collapsed or partly collapsed freeze dried cake, should be avoided. Preferably the freeze dried cake should have a volume corresponding to the (fill) volume of the formulation prior to freeze drying.

Freeze dried formulations should preferably form a stable homogeneous, nice appearing, and/or fluffy/porous freeze drying cake (such properties are often referred to as a “pharmaceutically elegant” freeze dried cake—a concept that is well known to the skilled person). Freeze dried formulations should furthermore preferably be easy to reconstitute, and the dissolved FVIII protein should be stable during the “in use period” (the time frame between reconstitution and administration).

The inventors have herein provided a FVIII formulation for sc administration, wherein the amount of sodium salt is significantly reduced and the amount of carbohydrate or sugar (preferably a non-reducing di-saccharide, such as e.g. sucrose), and preferably also anti-oxidant (methionine in particular), is significantly increased compared to currently available FVIII iv formulations.

Decreasing the amount of sodium salt and at the same time decreasing the amount of sucrose/carbohydrate (in order to decrease osmolality) was found to induce FVIII aggregation (e.g. HMWP—high molecular weight protein) during freeze drying. However, it is herein shown that increasing the amount of sugar/carbohydrate (preferably sucrose) in a “low salt formulation” enabled preparation of an isotonic (or close to isotonic) formulation with low content of protein aggregates, (measured as HMWP % by SE-HPLC) and low increase in aggregation during freeze drying and storage. High sucrose content contributed to a stabilizing matrix for FVIII, and resulted in a pharmaceutically elegant freeze dried cake with advantageous properties. Furthermore high sucrose concentration was found to stabilise the FVIII molecule after reconstitution.

However, the chemical stability was found to be reduced in the formulations with high concentration of sucrose and low concentration of salt. This problem was observed as increase in the content of oxidized FVIII light chain. The inventors were able to lower the amount of oxidised FVIII by addition of methionine to the sucrose based formulation, and by including degassing as part of the process.

The normal freeze drying process includes exposure of frozen aqueous samples to low pressure/vacuum conditions whereby the frozen water is sublimated (solid phase→gas phase) and gasses (including oxygen and water) are removed. Upon pressure equilibration, the gasses present in the vials may thus be exchanged with nitrogen. It has thus generally been assumed that freeze drying per se is sufficient to limit protein oxidation of the freeze dried product.

The inventors have herein surprisingly discovered that degassing a low salt/high sucrose FVIII formulation (optionally comprising about 1-10 mg/mL methionine) in the freeze dryer prior to the freezing step of the freeze drying process, results in improved FVIII stability with regards to (e.g.) oxidation (in particular oxidation of the FVIII light chain) during storage. Degassing prior to the freeze drying process appears to have no compromising effects with regards to the physical stability (aggregation propensity and chain dissociation) of FVIII. Thus stable freeze dried FVIII formulations (providing stable and active FVIII) can be prepared by a freeze-drying process including degassing step(s).

Degassing/removal of oxygen before freezing is preferably performed in a freeze dryer by applying low pressure (e.g. 100 mbar) for about 5-60 minutes (e.g. 20 minutes) at a temperature below 40° C., such as e.g. at about 4-5° C. or at room temperature (e.g. 20° C.). The pressure is equilibrated to about 1 atm (1013 mbar) with an inert gas such as nitrogen. This degassing procedure is preferably performed once, preferably twice, three times, four times or five times prior to the freezing step in order to improve FVIII stability and/or to reduce oxidation of FVIII.

### Definitions

Isotonic normally means that there is little or no osmotic pressure gradient between two solutions separated by a water permeable membrane—e.g. a cell membrane. Human plasma has an osmolality of about 300 mOsm/kg, thus isotonic solutions in general has an osmolality of about 300 mOsm/kg, and hence an osmolarity of about 300 mOsm/L solution (osmolality and osmolarity have similar values at low excipient concentrations). The osmolality is directly correlated to the chemical potential of water, and to the molal concentration of solutes e.g. sugar, protein, amino acid, dissociated electrolytes. Osmolality is a basic physical property of water/aqueous solutions quantifying the effects of solute addition, and it can be determined by freeze point depression or by vapour pressure osmometry.

The main purpose of reducing the osmolality (and injection volume) of a sc formulation (compared to the present iv formulations) is to avoid or reduce unwanted injection site reactions. Literature searches suggests that solutions having an osmolality of about 280-450 mOsm/kg are perceived as isotonic in connection with sc administration and the term “isotonic formulations” as used herein thus encompasses formulations of about 280-450 mOsm/kg—such formulations are suitable for both iv and extravascular administration such as e.g. sc administration. The tolerability of solutions having osmolality>300 mOsm/kg is also dependent on injection volume: Higher injection volume increases the risks of injection site reactions, e.g. a solution with an osmolality of 600 mOsm/kg and a target injection volume of 0.5 mL can be regarded as safe. Isotonic formulations as used herein encompasses formulations of about 280-600 mOsm/kg, alternatively, 300-600, 400-600, 500-600, 300-500, 350-500, 400-500, 300-400, 320-400, 340-400, 350-400, 280-380, 300-380, 320-380, 340-380, 350-380, 280-360, 300-380, 300-360, 300-350, 320-380, 350-380, 280-600, 300-600, 400-600, 500-600, 280, 281, 282, 283, 284, 285, 286, 287, 288, 289, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 mOsm/kg.

Factor VIII:

Factor VIII (FVIII) is a large, complex glycoprotein that is primarily produced by endothelial cells including liver sinusoidal endothelial cells (LSECs) and possibly also hepatocytes. Human FVIII codes for 2351 amino acids, including a signal peptide, and contains several distinct domains as defined by homology. There are three A-domains, a unique B-domain, and two C-domains. The domain order can be listed as NH2-A1-A2-B-A3-C1-C2-COOH. Small acidic regions C-terminal of the A1 (the a1 region) and A2 (the a2 region) and N-terminal of the A3 domain (the a3 region) play important roles in FVIII interaction with other coagulation proteins, including thrombin and von Willebrand factor (VWF).

During cellular processing, Furin cleaves prior to the a3 region. The resulting A1-a1-A2-a2-B chain is termed the heavy chain (HC) while the a3-A3-C1-C2 is termed the light chain (LC). The chains are connected by bivalent metal ion-bindings.

TABLE 1
FVIII domains and regions. The numbering of domains,
regions and single amino acid residues in the Factor VIII molecule
follow the numbering of full length Factor VIII (also if the B-domain
is truncated or if a fusion partner is added to the molecule).

| | | | Number of amino acid |
|---|---|---|---|
| Domain | Region | Amino acid number *) | residues |
| A1 | | 1-336 | 336 |
| | a1 | 337-372 | 36 |
| A2 | | 373-710 | 338 |
| | a2 | 711-740 | 30 |
| B **) | | 741-1648 | 908 |
| ***) | a3 | 1649-1689 | 41 |
| A3 | | 1690-2020 | 331 |
| C1 | | 2021-2173 | 153 |
| C2 | | 2174-2332 | 159 |
| Total FVIII | | 1-2332 | 2332 |

*) The numbering of domains, regions and single amino acid residues is in accordance with uniprot: P00451. Other FVIII alleles with FVIII activity exist as well in human populations and are also part of the present invention.
**) The nucleotide sequence encoding full length Factor VIII encodes a B domain of 908 amino acid residues. During protein synthesis, the B-domain in full-length FVIII is processed, resulting in a mixture of heavy chain with different length of B-domains attached (Jankowski MA et al. Haemophilia 2007; 13: 30-37). rFVIII with truncated B domains may comprise B domains being significantly shorter than 908 amino acids - one example of a truncated B domain is the 21 amino acid B domain linker according to SEQ ID NO 2.
***) Some naturally occurring FVIII variants comprise an a3 region spanning amino acids 1655-1689 and 1658-1689 (Lind P et al. Eur J Biochem 1995; 232: 19-27). Such FVIII proteins, as well as other naturally occurring FVIII variants, are also part of the present invention.

Endogenous FVIII molecules circulate in vivo as a pool of molecules with B domains of various sizes, the shortest having C-terminal at position 740, i.e. at the C-terminal of A2-a2, and thus contains no B domain. FVIII molecules with B-domains of different length all maintain procoagulant activity. Upon activation with thrombin, FVIII is cleaved at the C-terminal of A1-a1 at position 372, C-terminal of A2-a2 at position 740, and between a3 and A3 at position 1689, the latter cleavage releasing the a3 region with concomitant loss of affinity for VWF. The activated FVIII molecule is termed FVIIIa. The activation allows interaction of FVIIIa with phospholipid surfaces like activated platelets, and with activated factor IX (FIXa), i.e. the tenase complex is formed, allowing efficient activation of factor X (FX) resulting in thrombin generation and ultimately formation of a fibrin-stabilized haemostatic clot.

“Wildtype (wt)/native FVIII” is the human FVIII molecule derived from the full length sequence as shown in SEQ ID NO: 1 (amino acid 1-2332)—including allelic variants thereof. Deletion/truncation of the B domain is often considered to be an advantage for recombinant production of FVIII.

SEQ ID NO: 1: wt human FVIII (Ser750 residue shown
in bold and underline)
ATRRYYLGAVELSWDYMQSDLGELPVDARFPPRVPKSFPFNTSVVYKKTL
FVEFTDHLFNIAKPRPPWMGLLGPTIQAEVYDTVVITLKNMASHPVSLHA
VGVSYWKASEGAEYDDQTSQREKEDDKVFPGGSHTYVWQVLKENGPMASD
PLCLTYSYLSHVDLVKDLNSGLIGALLVCREGSLAKEKTQTLHKFILLFA
VFDEGKSWHSETKNSLMQDRDAASARAWPKMHTVNGYVNRSLPGLIGCHR
KSVYWHVIGMGTTPEVHSIFLEGHTFLVRNHRQASLEISPITFLTAQTLL
MDLGQFLLFCHISSHQHDGMEAYVKVDSCPEEPQLRMKNNEEAEDYDDDL
TDSEMDVVRFDDDNSPSFIQIRSVAKKHPKTWVHYIAAEEEDWDYAPLVL
APDDRSYKSQYLNNGPQRIGRKYKKVRFMAYTDETFKTREAIQHESGILG
PLLYGEVGDTLLIIFKNQASRPYNIYPHGITDVRPLYSRRLPKGVKHLKD
FPILPGEIFKYKWTVTVEDGPTKSDPRCLTRYYSSFVNMERDLASGLIGP
LLICYKESVDQRGNQIMSDKRNVILFSVFDENRSWYLTENIQRFLPNPAG
VQLEDPEFQASNIMHSINGYVFDSLQLSVCLHEVAYWYILSIGAQTDFLS
VFFSGYTFKHKMVYEDTLTLFPFSGETVFMSMENPGLWILGCHNSDFRNR
GMTALLKVSSCDKNTGDYYEDSYEDISAYLLSKNNAIEPRSFSQNSRHPS
TRQKQFNATTIPENDIEKTDPWFAHRTPMPKIQNVSSSDLLMLLRQSPTP
HGLSLSDLQEAKYETFSDDPSPGAIDSNNSLSEMTHFRPQLHHSGDMVFT
PESGLQLRLNEKLGTTAATELKKLDFKVSSTSNNLISTIPSDNLAAGTDN
TSSLGPPSMPVHYDSQLDTTLFGKKSSPLTESGGPLSLSEENNDSKLLES
GLMNSQESSWGKNVSSTESGRLFKGKRAHGPALLTKDNALFKVSISLLKT
NKTSNNSATNRKTHIDGPSLLIENSPSVWQNILESDTEFKKVTPLIHDRM
LMDKNATALRLNHMSNKTTSSKNMEMVQQKKEGPIPPDAQNPDMSFFKML
FLPESARWIQRTHGKNSLNSGQGPSPKQLVSLGPEKSVEGQNFLSEKNKV
VVGKGEFTKDVGLKEMVFPSSRNLFLTNLDNLHENNTHNQEKKIQEEIEK
KETLIQENVVLPQIHTVTGTKNFMKNLFLLSTRQNVEGSYDGAYAPVLQD
FRSLNDSTNRTKKHTAHFSKKGEEENLEGLGNQTKQIVEKYACTTRISPN
TSQQNFVTQRSKRALKQFRLPLEETELEKRIIVDDTSTQWSKNMKHLTPS
TLTQIDYNEKEKGAITQSPLSDCLTRSHSIPQANRSPLPIAKVSSFPSIR
PIYLTRVLFQDNSSHLPAASYRKKDSGVQESSHFLQGAKKNNLSLAILTL
EMTGDQREVGSLGTSATNSVTYKKVENTVLPKPDLPKTSGKVELLPKVHI
YQKDLFPTETSNGSPGHLDLVEGSLLQGTEGAIKWNEANRPGKVPFLRVA
TESSAKTPSKLLDPLAWDNHYGTQIPKEEWKSQEKSPEKTAFKKKDTILS
LNACESNHAIAAINEGQNKPEIEVTWAKQGRTERLCSQNPPVLKRHQREI
TRTTLQSDQEEIDYDDTISVEMKKEDFDIYDEDENQSPRSFQKKTRHYFI
AAVERLWDYGMSSSPHVLRNRAQSGSVPQFKKVVFQEFTDGSFTQPLYRG
ELNEHLGLLGPYIRAEVEDNIMVTFRNQASRPYSFYSSLISYEEDQRQGA
EPRKNFVKPNETKTYFWKVQHHMAPTKDEFDCKAWAYFSDVDLEKDVHSG
LIGPLLVCHTNTLNPAHGRQVTVQEFALFFTIFDETKSWYFTENMERNCR
APCNIQMEDPTFKENYRFHAINGYIMDTLPGLVMAQDQRIRWYLLSMGSN
ENIHSIHFSGHVFTVRKKEEYKMALYNLYPGVFETVEMLPSKAGIWRVEC
LIGEHLHAGMSTLFLVYSNKCQTPLGMASGH1RDFQITASGQYGQWAPKL
ARLHYSGSINAWSTKEPFSWIKVDLLAPMIIHGIKTQGARQKFSSLYISQ
FIIMYSLDGKKWQTYRGNSTGTLMVFFGNVDSSGIKHNIFNPPIIARYIR
LHPTHYSIRSTLRMELMGCDLNSCSMPLGMESKAISDAQITASSYFTNMF
ATWSPSKARLHLQGRSNAWRPQVNNPKEWLQVDFQKTMKVTGVTTQGVKS
LLTSMYVKEFLISSSQDGHQWTLFFQNGKVKVFQGNQDSFTPVVNSLDPP
LLTRYLRIHPQSWVHQIALRMEVLGCEAQDLY

The terms “B domain truncated” and “B domain deleted” (BDD) FVIII are used interchangeably herein. The B domain in FVIII spans amino acids 741-1648 of SEQ ID NO: 1. The B domain undergoes endo-proteolysis at several different sites, generating large heterogeneity in circulating plasma FVIII molecules as explained above and in Jankowski et al, Haemophilia 2007; 13: 30-37 and D'Amici et al, Electrophoresis 2010; 31: 2730-2739. While the B-domain plays a role in intracellular expression of FVIII, the exact extracellular function of the heavily glycosylated B domain, if any, is unknown. What is known is that the B domain is dispensable for FVIII activity in the coagulation cascade. Recombinant FVIII is thus frequently produced in the form of B domain-deleted/truncated variants. In one embodiment, the FVIII protein can be produced by an expression vector encoding a FVIII molecule comprising a 21 amino acid residue linker (B domain linker) sequence with the following sequence: SEQ ID NO 2: SFSQNSRHPSQNPPVLKRHQR. An O-glycan is attached to the underlined S in SEQ ID NO 2—this residue corresponds to position S750 in SEQ ID NO1. In another embodiment, the FVIII protein herein comprises a linker sequence with the following sequence: SEQ ID NO: 3: SFSQNSRHPSQNPPVLKRHQ. In another embodiment, the FVIII protein herein comprises a linker sequence with the following sequence: SEQ ID NO: 4: FSQNSRHPSQNPPVLKRHQR. In another embodiment, the FVIII protein herein are B domain deleted/truncated FVIII variants comprising an O-glycan attached to the Ser 750 residue shown in SEQ ID NO 1. A number of other O-glycans are thought to be attached to the B domain of the FVIII molecule but the exact location of these other O-glycans have not yet been determined.

FVIII Having an Increased In Vivo Circulatory Half-Life (Long Acting FVIII):

FVIII molecules according to the invention are long acting FVIII proteins—usually recombinant proteins that are e.g. fused to a fusion partner, conjugated to a half-life extending moiety, etc. in order to achieve a prolonged in vivo circulatory half-life of FVIII (“long acting FVIII”). The in vivo half-life of wt FVIII is about 12-14 hours—FVIII molecules according to the invention (long acting FVIII) have an in vivo circulatory half-life that is extended by (at least) 10%, preferably (at least) 15%, more preferably (at least) 20%, more preferably (at least) 25%, more preferably (at least) 30%, more preferably (at least) 40%, more preferably (at least) 50%, more preferably (at least) 60%, more preferably (at least) 70%, more preferably (at least) 80%, more preferably (at least) 90%, more preferably (at least) 100%. In vivo circulatory half-life can be e.g. measured in a suitable animal model.

“Half-life extending moieties” are sometimes referred to as “side chains”, “substituent”, etc. FVIII molecules having an increased in vivo circulatory half-life are sometimes also referred to as “protracted FVIII molecules” or “long acting FVIII molecules”. Half-life extending moieties include various types of polypeptides, peptidic compounds, polymeric compounds, water soluble polymers such as e.g. poly ethylen glycol (PEG), poly sialic acid (PSA), polysaccharides (e.g. dextran, starch, heparosan, etc.). The half-life extending moiety may alternatively be mainly hydrophobic in nature and include lipophilic components such as e.g. fatty acids, difatty acids, etc. (lipophilic moieties are sometimes referred to as “albumin binders”). The half-life extending moiety may furthermore comprise a linker between the FVIII molecule and the half-life extending moiety.

Long acting FVIII molecules can also be fused to a fusion partner via recombinant methods or via chemical/enzymatic conjugation. Examples of fusion partners include albumin, antibody Fc domains, Fc receptors, FVIII B domain fragments, synthetic peptides etc. Half-life conjugating moieties can e.g. be attached to glycans present in the FVIII molecules using chemical and/or enzymatic methods. Several glycans are present in FVIII, in particular in the B domain. In one embodiment, half-life extending moieties are conjugated to a B domain deleted/truncated FVIII molecule via an O-linked glycan attached to the S750 residue according to the amino acid numbering in SEQ ID NO 1. Preferably, the half-life conjugating moiety is conjugated to FVIII using enzymatic glyco-conjugating methods as disclosed in e.g. WO2009108806.

Amino acid sequences of FVIII fusion partners according to the present invention:

SEQ ID no. 5 (FVIII amino acids 741-966) ″226″
amino acid FVIII B domain:
SFSQNSRHPSTRQKQFNATTIPENDIEKTDPWFAHRTPMPKIQNVSSSDL
LMLLRQSPTPHGLSLSDLQEAKYETFSDDPSPGAIDSNNSLSEMTHFRPQ
LHHSGDMVFTPESGLQLRLNEKLGTTAATELKKLDFKVSSTSNNLISTIP
SDNLAAGTDNTSSLGPPSMPVHYDSQLDTTLFGKKSSPLTESGGPLSLSE
ENNDSKLLESGLMNSQESSWGKNVSS
SEQ ID NO 6-Human serum albumin:
DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVIDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRLA
KTYETTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASQAALGL
SEQ ID NO 7: extracellular region of human FcγRI
(CD64):
QVDTTKAVITLQPPWVSVFQEETVTLHCEVLHLPGSSSTQWFLNGTATQT
STPSYRITSASVNDSGEYRCQRGLSGRSDPIQLEIHRGWLLLQVSSRVFT
EGEPLALRCHAWKDKLVYNVLYYRNGKAFKFFHWNSNLTILKTNISHNGT
YHCSGMGKHRYTSAGISVTVKELFPAPVLNASVTSPLLEGNLVTLSCETK
LLLQRPGLQLYFSFYMGSKTLRGRNTSSEYQILIARREDSGLYWCEAATE
DGNVLKRSPELELQVLGLQLPTP
SEQ ID NO 8: Human IgG1 Fc domain:
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK
SEQ ID NO 9: The C-terminal 28 amino acids of the
beta-chain of human chorion gonadotropin (hCG C-
terminus):
SSSSKAPPPSLPSPSRLPGPSDTPILPQ
SEQ ID NO 10: Sequence A/XTEN (repetetive
sequences with varying lengths can be used):
GSPAGSPTSTEEGTSESATPESGPGTSTEPSEGSAPGSPAGSPTSTEEGT
STEPSEGSAPGTSTEPSEGSAPGTSESATPESGPGSEPATSGSETPGSEP
ATSGSETPGSPAGSPTSTEEGTSESATPESGPGTSTEPSEGSAPGTSTEP
SEGSAPGSPAGSPTSTEEGTSTEPSEGSAPGTSTEPSEGSAPGTSESATP
ESGPGTSTEPSEGSAPGTSESATPESGPGSEPATSGSETPGTSTEPSEGS
APGTSTEPSEGSAPGTSESATPESGPGTSESATPESGPGSPAGSPTSTEE
GTSESATPESGPGSEPATSGSETPGTSESATPESGPGTSTEPSEGSAPGT
STEPSEGSAPGTSTEPSEGSAPGTSTEPSEGSAPGTSTEPSEGSAPGTST
EPSEGSAPGSPAGSPTSTEEGTSTEPSEGSAPGTSESATPESGPGSEPAT
SGSETPGTSESATPESGPGSEPATSGSETPGTSESATPESGPGTSTEPSE
GSAPGTSESATPESGPGSPAGSPTSTEEGSPAGSPTSTEEGSPAGSPTST
EEGTSESATPESGPGTSTEPSEGSAPGTSESATPESGPGSEPATSGSETP
GTSESATPESGPGSEPATSGSETPGTSESATPESGPGTSTEPSEGSAPGS
PAGSPTSTEEGTSESATPESGPGSEPATSGSETPGTSESATPESGPGSPA
GSPTSTEEGSPAGSPTSTEEGTSTEPSEGSAPGTSESATPESGPGTSESA
TPESGPGTSESATPESGPGSEPATSGSETPGSEPATSGSETPGSPAGSPT
STEEGTSTEPSEGSAPGTSTEPSEGSAPGSEPATSGSETPGTSESATPES
GPGTSTEPSEGSAPG

Bioactivity:

FVIII molecules included in the formulation according to the present invention are capable of functioning in the coagulation cascade in a manner that is functionally similar, or equivalent, to human FVIII, inducing the formation of FXa via interaction with FIXa on an activated platelet and supporting the formation of a blood clot. FVIII activity can be assessed in vitro using techniques well known in the art. Clot analyses, FX activation assays (often termed chromogenic assays), thrombin generation assays and whole blood thrombo-elastography are examples of such in vitro techniques. FVIII molecules for use in a formulation of the present invention may have a specific FVIII activity that is at least about about 20%, at least about 30%, at least about 40%, at least about 50%, at least about 60%, at least about 70%, at least about 80%, at least about 90%, 100% or even more than 100% of that of native human FVIII when compared to e.g. human FVIII in e.g. a chromogenic assay (FVIII activity assay).

Degradation of FVIII

Factor VIII present in FVIII formulations can be degraded by several mechanisms, including oxidation, aggregation as well as dissociation between the two non-covalently associated protein subunits: the heavy chain (HC) and the light chain (LC).

Oxidation of FVIII (e.g. conjugated B domain deleted FVIII) can be measured by Reverse Phase High Performance Liquid Chromatography (RP-HPLC). During RP-HPLC, non-covalent interactions are unstable, hence LC and HC elute as separate peaks. The observed LC oxidation is primarily due to oxidation of methionine residues present in the light chain. Oxidized LC is detected as a separate peak in the chromatogram. LC oxidation is quantified as percentage of oxidized light chain (LC) compared to the total amount of FVIII protein (also referred to herein as oxidized LC %, oxidized forms %, or ox. forms %).

Aggregation of FVIII (e.g. conjugated B domain deleted/truncated FVIII) can be assessed by well-known techniques, e.g. by means of Size Exclusion High Performance Liquid Chromatography (SE-HPLC). Aggregated FVIII is detected by SE-HPLC as one peak or two peaks with lower retention time than the main peak (containing monomeric FVIII consisting of associated HC-LC). Aggregated FVIII is quantified by integration of this peak/these peaks as HMWP % (high molecular weight protein %) compared to the total area of peaks in the chromatogram. HC-LC dissociation can also be detected by mean of SE-HPLC, since free LC occurs as a separate peak eluting after the main peak.

It should be noted that FVIII degradation (primarily oxidation and aggregation) may occur during all process (e.g. handling and freeze drying) and continue after freeze drying—and during storage of pharmaceutical compositions herein. The compositions according to the invention should preferably have a content of degraded FVIII of less than, or no more than 10%, 9%, 8%, 7%, 6%, 5%, 4%,—after freeze drying. 15%, 14%, 13%, 12%, 11% after storage 2 years at 5 C, 12 months at 30 C, 3 months at 40 C. The composition according to the invention should preferably have a degradation rate of less than one percentage point per month for oxidized forms (oxidized FVIII LC), and 0.5 percentage point for HMWP (protein aggregation) at 30 C.

Cool storage conditions (e.g. 0° C., 1° C., 2° C., 3° C., 4° C., 5° C., 6° C., 7° C., 8° C., 9° C., 10° C., 0-10° C., 0-5° C., or 0-4° C.) favour a longer shelf life (e.g. up to one or two years or more) of the compositions according to the present invention. The shelf life of the pharmaceutical compositions according to the invention is at least 3 months at room temperature (25-30° C.).

Pharmaceutical Formulation:

Pharmaceutical formulations herein comprise various chemical substances/excipients, including long acting FVIII, and constitute a final medicinal product. The pharmaceutical formulations herein are aqueous formulations meaning that they comprise at least 75% water, preferably at least 80% water, preferably at least 85% water, preferably at least 90%, 91%, 92, 93%, 94%, 95%, 96% water (% w/w) after reconstitution of the freeze dried formulation in water or an aqueous solution (e.g. buffer).

The formulations herein are thus lyophilized/freeze dried formulations that are reconstituted prior to administration to the patient in need thereof. Reconstitution can take place at virtually any point in time prior to administration—but in most embodiments, reconstitution takes place, one day or less than one day in advance, 12 hours or less than 12 hours in advance, 6 hours or less than 6 hours in advance, 5 hours or less than 5 hours in advance, 4 hours, or less than 4 hours in advance, 3 hours or less than 3 hours in advance, 2 hours or less than 2 hours in advance, 1 hour or less than 1 hour in advance, 30 minutes or less than 30 minutes in advance, 20 minutes or less than 20 minutes in advance, 10 minutes or less than 10 minutes in advance, or 5 minutes or less than 5 minutes in advance of administration of the formulation to the patient. The formulations may thus be a formulation that has been reconstituted in aqueous solution/water/buffer prior to purchase or hand-over at a pharmacy, clinic, or hospital. The reconstituted formulation is preferably kept at low temperature (e.g. at or below 40° C., 30° C., 25° C., 20° C., 15° C., 10° C., 5° C., 4° C., 3° C., 2° C., 1° C.).

Following reconstitution, the formulations provided herein are suitable for use as parenteral formulations intended for e.g. intravenous or extravascular administration (e.g. intra-muscular, inter-dermal, and subcutaneous administration). As described herein, certain advantages are associated with use of the formulations herein for extravascular (preferably subcutaneous) administration.

One vial of a pharmaceutical formulation according to the present invention is preferably used as a single dosage administration in a patient. In connection with subcutaneous administration, one dosage pr. patient pr. day is preferably used in order to provide a relatively stable trough level of FVIII using a simple, convenient, and nearly pain-free regimen. Other dose regimens can however be employed e.g. once weekly, twice weekly, every second day, every third day, twice daily, three times daily, etc.) and the dosis regimen may also be adjusted according to specific needs of the patient—e.g. periods of increased/decreased physical activity, physical condition.

The concentration of long acting Factor VIII in the (reconstituted) formulation of the present invention is typically in the range of about 250-10,000 IU FVII/mL, 1000-10,000, 2000-10,000, 3000-10,000, 4000-10,000, 5000-10,000, 6000-10,000, 7000-10,000, 8000-10,000, or 500-10,000, or 500-5000 IU FVII/mL, such as e.g. 1000-3000, 1500-3000, 2000-3000, 2500-3000, 2000-3000, 2500-3000, 1000-2500, 1000-2000, 500-2500, 500-2000, 250-3000, 250-4000, 250-5000, 250-6000, 500-6000, 1000-6000, 2000-6000, 3000-40000, 500-8000, 500-7000, or 5000-6000 IU FVII/mL. Preferably, the concentration of Factor VIII in the formulation is 250, 300, 350, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000, 3500, 4000, 4500, 5000 5500, 6000, 6500, 7000, 7500, 8000, 8500, 9000, 9500, or 10,000 IU FVII/mL.

The concentration of FVIII can also be measured in g FVII/mL but a measurement of FVIII activity/mL more accurately reflects the effective amount of active ingredient. One IU/U (International Unit/Unit) is defined as the amount of (active) FVIII found in 1 mL of fresh, pooled normal human plasma. The terms “IU” and “U” are used interchangeably herein.

Often, one vial corresponds to one dose herein. Often the concentration or FVIII strength herein is denoted as U/mL if the fill volume in vials prior to freeze drying is lower than 1 mL the strength pr vial will thus be lower.

Salt:

The formulations according to the present invention comprise sodium salt and calcium salt, preferably NaCl and CaCl₂, 2H₂O. The formulations according to the present invention have a low total salt concentration: about 3-12, mg/mL (preferably about 5-10 mg/mL) in the reconstituted solution. Alternatively, the total salt content is about 3-11, 3-10, 3-9, 3-8, 3-7, 3-6, 3-5, 3-4, 4-11, 4-10, 4-9, 4-8, 4-7, 4-6, 4-5, 5-11, 5-10, 5-9, 5-8, 5-7, 5-6, 6-11, 6-10, 6-9, 6-8, 6-7, 7-11, 7-10, 7-9, 7-8, 8-11, 8-10, 8-9, 9-11, 9-10, or 10-11 mg total salt/mL in the reconstituted solution—alternatively mg total salt/mL (or mg salt/vial (dosage unit)).

The sodium salt is preferably NaCl present in an amount of about 1-10 mg/mL, such as e.g. 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 mg NaCl/mL. Preferably, the concentration of NaCl is about 1.0, 1.5, 2.0, 2.5, 3.0, 3.5, 4.0, 4.5, 5.0, 5.5, 6.0, 6.5, 7, 7.5, 8.0, 8.5, 9.0, 9.5 or 10.0 mg NaCl/mL. The molar weight of NaCl is 58.44 g per mole and 1-10 mg/mL NaCl thus correspond to about 17-171 mM. If a fill volume of less than 1 ml is used, then the total amount of NaCl/vial can be calculated easily—if e.g. NaCl is present in an amount of 5 mg/ml and the fill volume in the vial is about 0.5 ml, then the amount of NaCl pr. vial is about 2.5 mg/vial or mg/dose.

NaCl is known to have a solubilizing and stabilizing effect on FVIII and NaCl is therefore used in current FVIII formulations in relatively high concentrations. Another advantage of NaCl is that relatively high quantities thereof can be administered parenterally without causing any side effects—in contrast to e.g. potassium salts that can be toxic even in relatively low concentrations.

The calcium salt (preferably CaCl₂, 2H₂O) is present in the formulations herein in an amount of about 0.5-5.0 mg/mL such as e.g., 0.50, 0.55, 0.60, 0.65, 0.70, 0.75, 0.80, 0.85, 0.90, 0.95, or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8. 4.9, 5.0, 0.5-5, 1-5, 1.5-5, 2-5, 2.5-5, 3-5, 3.5-5, 4-5, 0.5-4, 1-4, 1.5-4, 2-4, 2.5-4, 3-4, 0.5-3, 1-3, 1.5-3, or 2-3 mg CaCl₂-2H₂O/mL. The molar weight of CaCl₂, 2H₂O is 147.03 g/mole and 0.5-5 mg CaCl₂, 2H₂O/mL thus corresponds to 3.4-34 mM If a fill volume of less than 1 ml is used, then the total amount of CaCl₂.2H₂O/vial can be calculated easily—if e.g. CaCl₂-2H₂O is present in an amount of 5 mg/ml and the fill volume in the vial is about 0.5 ml, then the amount of CaCl₂, 2H₂O pr. vial is about 2.5 mg/vial or mg/dose.

The presence of a divalent cation, e.g. Ca²⁺, is important for stabilization of the non-covalent interaction between HC and LC, and for prevention of FVIII aggregation, and hence for maintaining FVIII activity. Alternative calcium salts could be used herein, e.g. Calcium acetate (CaOAc₂), and other salts known to the skilled person. It is shown herein that calcium salt concentrations lower than about 0.5 or 0.4 mg/mL are associated with increased aggregation.

Carbohydrates/Saccharides and Polyols:

The formulations herein comprise a relatively high concentration of carbohydrates or saccharides or sugar—in particular mono- and/or disaccharides but also (or alternatively) sugar alcohols and/or polysaccharides. Examples of monosaccharides include glucose (dextrose), fructose (levulose), galactose, mannose, etc. Examples of disaccharides herein include sucrose, lactose, and trehalose. Examples of polysaccharides include dextran, raffinose, stachyose, starch. Examples of sugar alcohols include mannitol, sorbitol, inositol, galactitol, dulcitol, xylitol, and arabitol), alditols (e.g. glycerol (glycerine), 1,2-propanediol, 25 (propylene-glycol), 1,3-propanediol, 1,3-butanediol), and polyethylene-glycol. Carbohydrates/sugars provide the main component in the freeze dried formulations herein as well as contributing to stabilization of FVIII in solution, during freezing, and during drying/water removal, and during storage. Not all sugar alcohols are suitable for FVIII formulations. For example high mannitol concentration may destabilize FVIII molecules during freeze drying. Increased amounts of protein aggregates were detected in freeze dried formulations containing high mannitol concentrations. This destabilizing effect of mannitol was observed to be counter-acted by a stabilizing excipient/carbohydrate e.g. sucrose.

The formulations herein comprise 30-110 mg carbohydrate/mL (30-100 mg sucrose/ml corresponds to 87-292 mM) such as e.g. 30-90, 30-85, 30-80, 30-75, 30-70, 30-60, 30-50, 40-100, 40-90, 40-80, 40-85, 40-75, 40-70, 40-60, 40-50, 50-100, 50-90, 50-85, 50-80, 50-75, 50-70, 50-60, 60-100, 60-90, 60-85, 60-80, 60-75, 60-70, 70-100, 70-90, 70-85, 70-80, 75-80, 40-110, 50-110, 60-110, 70-110, 80-110, 90-110, 100-110, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110 mg sucrose (carbohydrate)/mL in the reconstituted. The molar mass of sucrose is 342.30 g/mol. Other carbohydrates/sugars e.g. non-reducing disaccharides may also be present in the formulations according to the invention e.g. trehalose, which was found to prevent protein aggregation (aggregation of GP-BDD-FVIII) during stress studies. In other embodiments, sucrose is the only carbohydrate/sugar present in the formulations herein.

If fill volume of less than 1 ml is used, then the total amount of sucrose can be calculated easily—if e.g. sucrose is present with a concentration of 100 mg/ml and the fill volume in the vial is about 0.5 ml, then the amount of sucrose pr. vial is about 50 mg/vial or mg/dose.

Buffers:

The formulations herein may comprise a buffer/buffering system. The buffer may be part of the lyophilized composition/formulation and/or it may be added to the lyophilized formulation in connection with resuspension/reconstitution thereof. The buffering substance/system may be selected from the group consisting of benzoate, glycylglycine, histidine or derivatives of histidine, Hepes, glycine, tris(hydroxymethyl)-aminomethan (TRIS), bicine, tricine, aspartic acid, glutamic acid, or mixtures thereof. In one embodiment of the invention, the concentration of the buffering substance is 1 mM, 5 mM, 10 mM, 15 mM, 20 mM, 25 mM, 30 mM, 40 mM, 50 mM, 60 mM, 70 mM, 80 mM, 90 mM, 100 mM, 1-100 mM, such as, e.g., 1-50 mM or 1-25 mM or 1-20 mM or 5-20 mM or 5-15 mM. 10-20 mM, 10-30 mM.

In one embodiment of the invention, the formulation comprises histidine, preferably L-histidine. In one embodiment thereof, the concentration of histidine/L-histidine is 1-10 mg/mL (corresponding to 6.4-64.5 mM), such as e.g. 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, or 9-10 mg/mL in the reconstituted formulation. The molar mass of histidine is about 155 g/mol. Buffer solutions may also be used to reconstitute the freeze dried formulations herein. Histidine was found to prevent protein aggregation (aggregation of GP-BDD-FVIII) during stress studies. Other amino acids like arginine and glutamine, as well as other buffer agents like succinate, were, in contrast, observed to have no effect on GP-BDD-FVIII in these stress studies.

pH of formulation herein (following reconstitution) is about 6.0-7.0, 6.0-7.5, or 6.2-6.8, or 6.3-6.7, alternatively 6.0, 6.1, 6.2, 6.3, 6.4, 6.5, 6.6, 6.7, 6.8, 6.9, 7.0, 7.1, 7.2, 7.3, 7.4, 7.5. The formulations herein thus have a pH close to neutral which is desirable e.g. in connection with formulations intended for injection e.g. subcutaneous administration.

Antioxidants/Reducing Agents:

The formulations herein (may) comprise an antioxidant. Antioxidants are used to prevent or reduce protein oxidation during preparation, freeze drying or storage. In one embodiment of the invention, a reducing agent such as methionine (or other sulphuric amino acids or sulphuric amino acid analogues) may be added to inhibit/reduce oxidation (primarily of methionine residues to methionine sulfoxide). The amount to be added should be an amount sufficient to inhibit oxidation. In one embodiment of the invention, the formulation comprises methionine, e.g., L-methionine. In one embodiment thereof, the concentration of the methionine/L-methionine in the reconstituted formulation is 0.5-100 mM, or 1.5-100 mM, such as, e.g. 0.5-15, 0.5-10, 0.5-5, 1-15, 1-10, 1-5, 2.0-20.0, 5.0-20.0, 10-20, 15-20, 10-50, 15-50, 20-50, 20-100, 30-100, 50-100, 50-90, 50-80, 1.5-15, 2.0-15, 5-15, 10-15, 1.5-10.0, 2.0-10.0, 5-10, 1.5-5.0, 2.0-5.0, 0.5, 1.0, 1.5, 2.0, 2.5, 3.0, 3.5, 4.0, 4.5, 5.0, 6, 7, 8, 9, 10, 15, 20, 25, 30, 40, 50, 50, 60, 70, 80, 90, or 100 mM antioxidant such as e.g. methionine. 1.5-100 mM methionine corresponds to about 0.22-15 mg methionine/mL in the reconstituted formulation—alternatively g methionine/vial (dosage unit). The molar mass of methionine is about 149.21 g/mol. Alternative antioxidants could be used e.g. ascorbic acid.

Other Excipients:

The formulations herein may further contain additional excipients. Examples of standard excipients for use in a pharmaceutical formulation according to the present invention are preservative(s) such as phenol, cresol, m-cresol, benzyl alcohol and phenoxyethanol, and surfactant(s). The formulations herein are, however, preferably essentially devoid of any preservatives as the inventors have made the discovery that even small amounts of standard preservatives (e.g. cresol and phenol) may result in destabilization of the FVIII molecule.

Typical surfactants suitable for use herein (with trade names given in brackets [ ]) are polyoxyethylene sorbitan fatty acid esters such as polysorbate 20 [Tween 20], polysorbate 40 [Tween 40], polysorbate 80 [Tween 80], poloxamers such as polyoxypropylene-polyoxyethylene block copolymer [Pluronic F68/poloxamer 188], polyethylene glycol octylphenyl ether [Triton X-100] or polyoxyethyleneglycol dodecyl ether [Brij 35]. The use of a surfactant in pharmaceutical formulations is well-known to the skilled person. In connection with protein formulations, the use of a mild surfactant (e.g. a non-ionic surfactant) such as e.g. a polysorbate (e.g. Tween 20) is generally preferred. In one embodiment, a surfactant such as e.g. Tween 20, is present in amount of 0.00-1.00, 0.01-0.10, 0.01-0.05, 0.05-0.10, 0.05-1.00, 0.1-1.0, 0.2-1.0, 0.3-1.0, 0.4-1.0, 0.5-1.0, 0.6-1.0, 0-7-1.0, 0.8-1.0, 0.9-1.0, 0.05-0.80, 0.1-0.8, 0.2-0.8, 0.3-0.8, 0.4-0.8, 0.5-0.8, 0.6-0.8, 0.7-0.8, 0.05-0.50, 0.1-0.5, 0.2, −0.5, 0.3-0.5, 0.4-0.5, 0.00, 0.05, 0.1, 0.2, 0.3, 0.4, 0.5, 0.6, 0.7, 0.8, 0.9, or 1.0 mg surfactant/mL in the reconstituted formulation. In connection with the present invention, relatively low amounts of surfactant 0.05-0.4 are preferred.

Anti-Oxidation/De-Gassing:

An antioxidant effect can be achieved by displacing oxygen (air) from contact with the formulations herein (de-gassing). De-gassing can be carried out with or without equilibration to e.g. atmospheric pressure before the start of the freeze drying process herein. The susceptibility of FVIII/long acting FVIII to oxidation can be fully or partly controlled by exclusion of atmospheric air or by displacing oxygen (air). This may be accomplished by saturating the liquid formulation with e.g. nitrogen, helium or argon before freezing and freeze drying. The displacement of oxygen (air) may e.g. be carried out as a “degassing” process where the solution is subjected to one or more cycles of (i) exposure to an inert gas (argon, helium or nitrogen) and/or (ii) exposure to low pressure, a pressure below atmospheric pressure. The formulations herein can be sterile filtered, distributed in vials, and degassed by e.g. exposing the vials to 0.1 bar (in the Freeze Dry/FD chamber) followed by pressure equilibration by N₂(g). One, two, three, four, or five cycles can be performed prior to sealing of vials (in the chamber) under N₂ (or another inert gas).

Alternatively, the formulations herein may be degassed by manufacturing the formulation in an oxygen-free atmosphere and by dissolving/reconstituting the excipients in oxygen-free water. This type of formulation is subsequently freeze dried and preferably stored under oxygen-free conditions by e.g. filling sealed vials with inert gas.

Use of an antioxidant may be combined with exclusion of atmospheric air/de-gassing. Furthermore, the formulations herein may be protected from light; combined with exclusion of atmospheric air and/or use of an antioxidant. Thus, the present invention also provides an air-tight container (e.g. a vial or a cartridge (such as a cartridge for a pen applicator)) containing the freeze dried or reconstituted formulation as defined herein, and optionally an inert gas. The inert gas may be selected from the group consisting of nitrogen, helium or argon. The term “air-tight container” means a container having a low permeability to oxygen (air). The container (e.g. vial or cartridge or syringe) is typically made of glass or plastic, in particular glass, optionally closed by a rubber septum or other closure means, allowing for penetration with e.g. a needle, with preservation of the integrity of the pharmaceutical formulation. In a further embodiment, the container is a vial or cartridge enclosed in a sealed bag, e.g. a sealed plastic bag, such as a laminated (e.g. metal (such as aluminium) laminated plastic bag).

The present invention also encompasses a method of treating haemophilia A, which method comprises administering a formulation according to the present invention to a subject in need thereof. The term “subject”, as used herein, includes any human patient, or non-human vertebrate. The term “treating” or “treatment”, as used herein, refers to the medical therapy of any human or other vertebrate subject in need thereof. Said subject is expected to have undergone physical examination by a medical practitioner, or a veterinary medical practitioner, who has given a tentative or definitive diagnosis which would indicate that the use of the formulations herein is beneficial to the health of said human or other vertebrate. The timing and purpose of such treatment may vary from one individual to another, according to the status quo of the subject's health. Thus, said treatment may be prophylactic, palliative, symptomatic and/or curative. In terms of the present invention, prophylactic, palliative, symptomatic and/or curative treatments may represent separate aspects of the invention.

The clinical severity of haemophilia A is determined by the concentration of functional units of FVIII in the blood and is classified as mild, moderate, or severe. Severe haemophilia is defined by a clotting factor level of <0.01 U/mL corresponding to <1% of the normal level, while moderate and mild patients have levels from 1-5% and >5%, respectively.

Protein Concentration Before and after Freeze Drying:

Volumes of the formulations herein before freeze drying (fill volume) and after freeze drying (reconstitution volume) may e.g. be 1:1, or close to 1:1. In one embodiment, the volumes before vs. after reconstitution may be about 1.0:1.1, 1.0-1.2, 1.0-1.2, 1.0-1.3, 1.0-1.4, 1.0-1.5, 1.0-1.6, 1.0-1.7, 1.0-1.8, 1.0-1.9, 1.0-2.0, 1.0-2.5, 1.0-3.0, 1:0.9, 1:0.8, 1:07, 1:0.6, 1:0.5, 1:0.4 or 1:0.3.

Freeze Dried Formulation:

Pharmaceutical formulations according to the invention are freeze dried formulations that are reconstituted in sterile water or aqueous solutions (e.g. buffer) prior to use. The freeze dried formulations herein, prior to reconstitution, appear homogeneous in structure and are fast and easily reconstituted (“pharmaceutically elegant” freeze dried cake). The matrix or bulk of the freeze dried cake mainly consists of the freeze dried excipients—the freeze dried matrix furthermore has a volume that essentially corresponds to the volume of the solution which is fill in the vial before freeze drying.

The total volume of the reconstituted formulation is very close to the volume of the reconstitution solution (buffer/water) and no adjustments of the total volume versus the reconstitution volume are therefore made herein. If e.g. the formulations herein are reconstituted in 1 ml buffer/water, then the total volume of the reconstituted formulation will be very close to 1 ml—e.g. about 1.01-1.05 ml—the calculations herein do therefore not take these minor differences into account.

### LIST OF EMBODIMENTS

It is understood that all aspects and embodiments of the invention can be combined and that they are not to be understood in any limiting way.

### Embodiment 1

A (freeze dried or reconstituted freeze dried) pharmaceutical FVIII formulation, wherein said formulation comprises a FVIII molecule having an increased in vivo circulatory half-life compared to wt FVIII (“Long acting FVIII)”), wherein said formulation, following reconstitution, is an aqueous isotonic (or close to isotonic) formulation, and wherein said formulation comprises 250-10.000 IU/mL of said FVIII molecule (preferably 1000, 1500, 2000, 2500, 3000, 4000, or 5000 IU/mL), 2-7 mg NaCl/mL, 0.5-5.0 mg CaCl₂, 2H₂O/mL, and 50-110 mg sucrose/mL. Alternatively, sucrose can be fully or partly replaced by trehalose in the formulations according to the present invention.

### Embodiment 2

A pharmaceutical formulation according to the invention, wherein said formulation further comprises 0.5-15, 0.5-10, 0.5-5, 1-5, or 2-4 mg histidine—alternatively mg histidine/mL following reconstitution. The pharmaceutical formulation according to the invention optionally furthermore comprises 0.5-15, 0.5-10, 0.5-5, 1-5, or 2-5 mg methionine (alternatively mg methionine/mL following reconstitution).

### Embodiment 3

A pharmaceutical formulation according to the invention, wherein said formulation further comprises 0.05-0.5 mg surfactant—alternatively 0.1-0.5 mg surfactant/mL following reconstitution. The surfactant is preferably a non-ionic (mild) surfactant such as e.g. Tween® 20.

### Embodiment 4

A pharmaceutical formulation according to the invention, wherein the volume of said reconstituted formulation is about 0.2-1.5 mL, preferably 0.3-1.5, preferably 0.4-1.5, preferably 0.5-1.5, preferably 0.5-1.0, preferably 0.5-1.2, preferably 0.4-1.0, preferably 0.4-1.2, preferably 0.8-1.2 mL, e.g. 0.2 mL, 0.3 mL, 0.4 mL, 0.5 mL, 0.6 mL, 0.7 mL, 0.8 mL, 0.9 mL, or 1 mL, and wherein the osmolality of the (reconstituted) formulation is about 280-400 or 300-500 mOsm/kg. The formulation may be reconstituted in (pure and/or sterile) water or buffer. Preferably, one dosage (preferably in a glass vial) of reconstituted pharmaceutical formulation according to the invention is used for one administration/injection in a patient.

### Embodiment 5

A pharmaceutical formulation according to the invention, wherein said FVIII molecule is a B domain truncated molecule comprising a B domain linker of 15-25 amino acids (preferably 17-22 amino acids, preferably 19-21 amino acids), wherein said FVIII molecule is conjugated with a half-life extending moiety via an O-glycan linked to a Serine amino acid residue corresponding to the Ser750 residue according to SEQ ID NO 1. Preferably, the sequence of the FVIII B domain linker is as set forth in SEQ ID NO 2, SEQ ID NO 3, or SEQ ID NO 4. Glyco-conjugation via the S750 residue may be performed using e.g. enzymatic or chemical methods. Enzymatic glyco-conjugation of a FVIII molecule is e.g. described in WO09108806.

### Embodiment: 6

A pharmaceutical formulation according to the invention, wherein said FVIII molecule is conjugated with a water soluble polymer.

### Embodiment 7

A pharmaceutical formulation according to the invention, wherein said water soluble polymer is PEG. The size of the PEG polymer is preferably about 20-100 kDa, more preferably about 30-50 kDa, such as e.g. 20, 30, 40, 50, 60, 70, 80, 90, or 100 kDa.

### Embodiment 8

A pharmaceutical formulation according to the invention, wherein said water soluble polymer is heparosan. The size of the heparosan polymer is preferably about 20-150 kDa, 50-150 kDa, 50-100 kDa, more preferably 30-50 kDa, more preferably 70-90 kDa, such as e.g. 20, 30, 40, 50, 60, 70, 80, 81, 82, 83, 84, 85, 86, 87, 88, 89, 90, 100, 120, 140, or 150 kDa.

### Embodiment 9

A pharmaceutical formulation according to the invention, wherein said FVIII molecule is a fusion protein (FVIII fused to a fusion partner).

### Embodiment 10

A pharmaceutical formulation according to the invention, wherein the fusion partner of said fusion molecule is selected from the list consisting of: albumin, an Fc domain, an Fc receptor, and a FVIII B domain fragment of about 200-400 amino acids.

### Embodiment 11

A pharmaceutical formulation according to the invention, wherein said formulation comprises 250-10,000 IU FVII/mL, 3.1 mg histidine/mL, 2.5 mg methionine/mL, 3.0 mg NaCl/mL, 80 mg sucrose/mL, 0.4 mg non-ionic surfactant/mL, and 3 mg CaCl₂, 2H₂O/mL.

### Embodiment 12

A pharmaceutical formulation according to the invention, wherein said formulation comprises 250-10,000 IU FVII/mL, 3.1 mg histidine/mL, 2.5 mg methionine/mL, 3.5 mg NaCl/mL, 70 mg sucrose/mL, 0.4 mg non-ionic surfactant/mL, and 0.5-1 mg CaCl₂, 2H₂O/mL.

### Embodiment 13

A pharmaceutical formulation according to the invention, wherein said formulation comprises 250-10,000 IU FVII/mL, 3.1 mg histidine/mL, 2.5 mg methionine/mL, 3.5 mg NaCl/mL, 90 mg sucrose/mL, 0.4 mg non-ionic surfactant/mL, and 2 mg CaCl₂, 2H₂O/mL.

### Embodiment 14

A pharmaceutical formulation according to the invention, wherein said formulation comprises 250-10,000 IU FVII/mL, 3.1 mg histidine/mL, 2.5 mg methionine/mL, 4 mg NaCl/mL, 100 mg sucrose/mL, 0.1-0.4 mg non-ionic surfactant/mL, and 2 mg CaCl₂, 2H₂O/mL.

### Embodiment 15

A pharmaceutical formulation according to the invention, wherein said formulation comprises 1000-10,000 IU FVII/mL, 1-4 mg histidine/mL, 2.5 mg methionine/mL, 7 mg NaCl/mL, 100 mg sucrose/mL, 0.4 mg non-ionic surfactant/mL, and 2 mg CaCl₂, 2H₂O/mL.

### Embodiment 16

A pharmaceutical formulation according to the invention, wherein said formulation comprises 250-10,000 IU FVII/mL, 1.5 mg/ml histidine, 2.5 mg methionine/mL, 3.5 mg NaCl/mL, 70 mg sucrose/mL, 0.4 mg non-ionic surfactant/mL, and 0.5-1 mg CaCl₂, 2H₂O/mL.

### Embodiment 17

A pharmaceutical formulation according to the invention, wherein said formulation, following reconstitution, comprises 2.5-3.5 mg histidine/mL, such as e.g. 3.1 mg histidine/mL.

### Embodiment 18

A pharmaceutical formulation according to the invention, wherein said formulation, following reconstitution, comprises 3-4 mg NaCl/mL, such as e.g. 3.5 mg NaCl/mL.

### Embodiment 19

A pharmaceutical formulation according to the invention, wherein said formulation, following reconstitution, comprises 0.3-1.0 mg CaCl₂, 2H₂O/mL, such as e.g. 0.5 mg CaCl₂, 2H₂O/mL.

### Embodiment 20

A pharmaceutical formulation according to the invention, wherein said formulation, following reconstitution, comprises 2-3 mg methionine/mL, such as e.g. 2.5 mg methionine/mL.

### Embodiment 21

A pharmaceutical formulation according to the invention, wherein said formulation, following reconstitution, comprises 60-80 mg or 70-80 mg sucrose/mL, such as e.g. 70 mg sucrose/mL.

### Embodiment 22

A pharmaceutical formulation according to the invention, wherein said formulation, following reconstitution, comprises 0.2-0.4 mg non-ionic (and/or mild) surfactant/mL, such as e.g. 0.4 mg Polysorbate 20 (Tween® 20)/mL.

### Embodiment 23

A pharmaceutical formulation according to the invention, wherein the amount of oxidized FVIII light chain molecules is below 10%, preferably below 9%, preferably below 8%, preferably below 7%, preferably below 6%, preferably below 5%, preferably below 4%, preferably below 3%, preferably below 2% or preferably below 1% of the total amount of FVIII. Preferably, the amount of oxidized FVIII light chain products are measured after storage for 3 months, 4 months, 5 months or 6 months at 20-30 degC.

### Embodiment 24

A pharmaceutical formulation according to the invention, wherein the formulation is reconstituted in a 0.5-15 mM (preferably 0.5-5 mM) histidine solution: The volume of the reconstituted formulation is preferably about 0.2 mL, 0.3 mL, 0.4 mL, 0.5 mL, 0.6 mL, 0.7 mL, 0.8 mL, 0.9 mL, 1.0 mL, 1.1 ml, 1.2 ml, 1.3 ml, 1.4 mL, or 1.5 mL.

### Embodiment 25

A pharmaceutical formulation according to the invention, wherein said formulation, following reconstitution, comprises 250-10.000 IU FVII/mL, 3.5 mg NaCl/mL, 0.5-1.0 mg CaCl₂ 2 H₂O/mL, 3.1 mg histidine/mL, 2.5 mg methionine/mL, 70 mg sucrose/mL, 0.4 mg Tween 20/polysorbate 20/mL, 350-400 mOsm/kg, wherein said formulation is reconstituted in 10 mM histidine solution.

### Embodiment 26

A pharmaceutical formulation according to the invention, wherein the freeze dried formulation, prior to reconstitution, is a pharmaceutically elegant freeze dried cake. The freeze dried cake is preferably placed on the bottom of a dosage vial, such as a glass vial. The volume of the freeze dried cake preferably corresponds essentially to the fill volume before freeze drying.

### Embodiment 27

A (freeze dried or reconstituted freeze dried) pharmaceutical FVIII formulation, wherein said formulation comprises a FVIII molecule having an increased in vivo circulatory half-life compared to wt FVIII, wherein said formulation is an aqueous isotonic formulation following reconstitution, and wherein said formulation comprises 250-10.000 IU/mL of said FVIII molecule (following reconstitution), a low NaCl concentration (e.g. 1-5 mg NaCl/mL following reconstitution), a high sugar/sucrose concentration (e.g. 60-80 mg sucrose/mL following reconstitution), about 0.5-1 mg/mL CaCl₂ (2H₂O) and wherein the amount of oxidized FVIII light chain molecules is below 10%, preferably 5%, preferably 1% of the total amount of FVIII. The total volume of the reconstituted formulation is preferably about 1 mL or less than 1 mL e.g. 0.3 or 0.5 mL. Preferably, the amount of oxidized FVIII light chain is below 5% after three months storage at 30 degC.

### Embodiment 28

A process for making a (freeze dried or reconstituted freeze dried) pharmaceutical formulation according to the invention, wherein said process comprises the step of degassing the liquid formulation by exposure of the (liquid) formulation to low pressure (significantly below 1 atm., e.g. 0.01-0.50 atm.) followed by pressure equilibration with an inert gas prior to freeze drying of the liquid formulation. The step of degassing the liquid formulation prior to freezing and freeze drying can be performed once, twice, three times, four times, or even five times or more for about 1-120 minutes, preferably 1-60 minutes, preferably 1-45 minutes, preferably 1-40 minutes, preferably 1-30 minutes, preferably 1-20 minutes, preferably 1-15 minutes, preferably 1-10 minutes, preferably 5-120 minutes, preferably 5-60 minutes, preferably 5-45 minutes, preferably 5-40 minutes, preferably 5-30 minutes, preferably 5-20 minutes, preferably 5-15 minutes, preferably 10-120 minutes, preferably 10-60 minutes, preferably 10-45 minutes, preferably 10-40 minutes, preferably 10-30 minutes, preferably 10-20 minutes, preferably 10-15 minutes, preferably 15-120 minutes, preferably 15-60 minutes, preferably 15-45 minutes, preferably 15-30 minutes, preferably 15-20 minutes, or preferably 20-40 minutes. The freeze dried formulation is reconstituted in water, or an aqueous solution/buffer, prior to administration to a patient. The formulation, and preferably also the solution used for reconstitution, has preferably been subject to a sterile filtration step prior to degassing and freeze drying.

### Embodiment 29

A process for making a (freeze dried or reconstituted freeze dried) pharmaceutical formulation according to the invention, wherein said process comprises the step of solubilizing the excipients (FVIII, sugar, salt, etc.) in water essentially devoid of oxygen (e.g. degassed water) followed by freeze drying of the resulting liquid formulation. This process preferably takes place in at atmosphere substantially without oxygen (e.g. N₂).

### Embodiment 30

A pharmaceutical formulation produced or obtained by, or obtainable by, the method according to the invention.

### Embodiment 31

A pharmaceutical formulation according to the invention, wherein said formulation is intended for extravascular, preferably subcutaneous administration. A formulation intended for subcutaneous administration is preferably administered once pr. month, twice pr. month, once pr. week, twice pr. week, once daily, twice daily, or three times daily.

### Embodiment 32

A pharmaceutical formulation according to the invention, wherein said formulation is intended for intravenous administration. A formulation intended for intravenous administration is preferably administered once pr. month, once every second week, once pr. week, twice pr. week, three times pr. week, once daily, twice daily, or three times daily—or on demand.

### Embodiment 33

A pharmaceutical formulation according to the invention, wherein said formulation is intended for once daily or once weekly administration.

### Embodiment 34

A pharmaceutical formulation according to the invention for use in treatment of haemophilia A.

### Embodiment 35

A method of treatment of haemophilia, preferably haemophilia A, wherein said method comprises administration of a pharmaceutical formulation according to the invention to a patient in need thereof.

### EXAMPLES

Throughout the examples herein, the following freeze dryers were used: Steris Lyovac FCM10, Usifroid SMH 45S or Genesis 25 LSQ EL-85. No differences in the appearance or stability of freeze dried formulations could be related to the type of freeze drying equipment (the type of freeze dryer).

### Example 1 Degassing Procedure

Degassing: Vials with liquid formulation were placed on the shelf of the freeze dryer and the shelf was cooled to 5° C. The pressure was then decreased to 100 mBar and this pressure was maintained for 20 minutes. The pressure was then increased to 900 mBar with nitrogen and this pressure was maintained for 20 minutes. Then the pressure was decreased to 100 mBar again and the pressure was maintained at 100 mBar for 20 minutes. The pressure was then increased to atmospheric pressure with nitrogen and the freeze drying was started.

The oxygen content in the formulation was about 320 micromolar/L before degassing. The oxygen content in the formulation after this degassing procedure (before the start of the freeze drying) was about 30 micromolar/L. The difference in oxygen concentration before and after degassing shows that the degassing procedure herein is an effective way of decreasing the oxygen content in a formulation.

The degassing procedure was also performed at room temperature without the initial cooling of the shelves with a similar result: The oxygen content was measured to about 30 micromolar/L after degassing.

### Example 2 Production of Tested Compound

Glyco-conjugated B domain truncated/deleted FVIII can be produced as disclosed e.g. in Example 1 in WO09108806.

### Example 3 Determination of Percentage of Oxidized Light Chain by Reverse Phase High Performance Liquid Chromatography (RP-HPLC)

The chemical stability of glycopegylated B-domain deleted Factor VIII (GP-BDD-FVIII produced according to example 2) was evaluated by RP-HPLC. The method was used to quantify the percentage of oxidized light chain (LC) compared to the total amount of protein in one sample. Oxidized LC % was used to compare the chemical stability of GP-BDD-FVIII produced under different conditions e.g. with and without degassing prior to freeze drying. Oxidized LC % was further used to compare the chemical stability of GP-BDD-FVIII in different formulations.

For reverse phase HPLC analysis, a Dimethylbutyldimethylsilane C4 column was used (DMeBuDMeSi, FEF Chemicals, Denmark). Pore size: 300 Å, Particle size: 5 m, Column dimensions 2.1×250 mm. Mobile phase A: 0.15% TFA, Mobile phase B: 0.14% TFA, 80% MeCN. Flow rate: 0.5 mL/min. Gradient: Time/% B: 0/35 28/80.5 29/100 34/100 35/35.

### Example 4 Determination of FVIII HMWP % by Size Exclusion High Performance Liquid Chromatography (SE-HPLC)

The physical stability, the aggregation propensity, of GP-BDD-FVIII was evaluated by SE-HPLC. The method was used to quantify the percentage of aggregated protein/high molecular weight protein (HMWP %) compared to the total amount of protein in one sample. HMWP % was used to compare the physical stability of GP-BDD-FVIII in various formulations. For SE-HPLC analyses (in example 6-14, 21) a Sepax Zenix, SEC-300 column was used. Pore size: 300 Å, column dimensions: 300×7.8 mm, elution buffer: 10 mM Bis-Tris propane, 500 mM NaCl, 10 mM Calcium Acetate, 10% 2-Propanole, pH 6.8, flow rate: 0.3 mL/min, run time 60 min. HMWP % is quantified as the percentage of the integrated peak area of peak/peaks, eluting prior to the main peak, compared to the total integrated peak area of the chromatogram.

For SE-HPLC analyses (in example 15-18, 20, 22-24) a Sepax, SRT SEC-500 column was used. Pore size: 500 Å, column dimensions: 300×7.8 mm, elution buffer: 10 mM Tris, 300 mM NaCl, 10 mM Calcium Chloride, 5% 2-Propanole, pH 7, flow rate: 0.3 mL/min, run time 60 min. HMWP % is quantified as the percentage of the integrated peak area of peak/peaks, eluting prior to the main peak, compared to the total integrated peak area of the chromatogram.

### Example 5 Concentration Method

The concentration of GP-BDD-FVIII can be determined based on the area of the main peak/the GP-BDD-FVIII monomer peak. This peak area in the SE-HPLC chromatogram is compared to a standard curve using reference material with a known concentration (determined by an orthogonal method). The SE-HPLC methods used for concentration determination are identical to the ones described in Example 4.

### Example 6 Effects of Degassing

A formulation containing glycopegylated B-domain deleted Factor VIII (GP-BDD-FVIII) was prepared with the following composition: 1000 IU/mL GP-BDD-FVIII, 70 mg/mL sucrose, 3.5 mg/mL NaCl, 0.5 mg/mL CaCl₂, 0.22 mg/mL methionine, 1.55 mg/mL L-histidine, 0.4 mg/mL polysorbate 20. The formulation was separated into vials. The vials were split into two groups: one group was exposed to degassing prior to freeze drying, and one group was freeze dried without preceding degassing. All vials were freeze dried using the same freeze drying program (see table 6.1).

Degassing procedure: Degassing took place in the freeze dryer prior to the freezing steps. Oxygen was removed from the liquid by applying low pressure (100 mbar) during 20 minutes at +20° C. The pressure was equilibrated to 1 atm (1013 mbar) by nitrogen gas. The degassing procedure was repeated before the freezing step.

TABLE 6.1
Freeze drying program

| | | Time | | Alarm | Pressure | R/H |
|---|---|---|---|---|---|---|
| Process step | Step | H:Min | ° C. | Pressure | hPa¹ | step |
| Load | 01 | — | +5 | — | Ambient | Hold |
| Equilibration | 02 | 01:00 | +5 | — | Ambient | Hold |
| Freezing | 03 | 01:00 | −45 | — | Ambient | Ramp |
| Freezing | 04 | 03:00 | −45 | — | Ambient | Hold |
| Annealing | 05 | 01:00 | −8 | — | Ambient | Ramp |
| Annealing | 06 | 12:00 | −8 | — | Ambient | Hold |
| Annealing | 07 | 01:00 | −45 | — | Ambient | Ramp |
| Annealing | 08 | 03:00 | −45 | — | Ambient | Hold |
| Pull vacuum | 09 | 00:15 | −45 | 0.1 | 0.05 | Ramp |
| Primary drying | 10 | 01:00 | −30 | 0.1 | 0.05 | Ramp |
| Primary drying | 11 | 45:00 | −30 | 0.1 | 0.05 | Hold |
| Secondary drying | 12 | 14:00 | +40 | 0.1 | 0.05 | Ramp |
| Secondary drying | 11 | 12:00 | +40 | 0.1 | .05 | Hold |

Total time: about 94 hours
¹MKS pressure gauge, capacitance manometer gauge

After freeze drying with and without preceding degassing the freeze dried formulations were reconstituted by 1.1 mL 10 mM histidine, pH 6.0, and analysed by RP-HPLC for oxidized forms (as described in example 3). The percentage of oxidized LC was determined right after freeze drying (at T=0), and after 3 weeks (storage of freeze dried formulations) at 30° C. and 40° C., see data in table 6.2.

TABLE 6:2
The percentage of oxidized LC in a freeze dried formulation of GP-BDD-
FVIII with- and without preceding degassing.

| | Formulation produced by | Formulation produced by |
|---|---|---|
| | freeze drying with | freeze drying without |
| Storage | preceding degassing | preceding degassing |
| Before freeze | 3.1% oxidized LC | 3.2% oxidized LC |
| drying | | |
| T = 0 | 3.0% oxidized LC | 4.0% oxidized LC |
| 3 weeks at 30° C. | 4.1% oxidized LC | 8.0% oxidized LC |
| 3 weeks at 40° C. | 4.7% oxidized LC | 9.9% oxidized LC |

Oxidation of GP-BDD-FVIII is thus significantly reduced upon degassing prior to freeze drying. Lower percentage of oxidized LC was detected at T=0 (right after freeze drying), and in particular after storage at 30° and 40° C. during 3 weeks (data shown in table 6.2)

### Example 7 Effects of Degassing

A formulation containing glycopegylated B-domain deleted Factor VIII (GP-BDD-FVIII) was prepared with the following composition: 1000 IU/mL GP-BDD-FVIII, 70 mg/mL sucrose, 3.5 mg/mL NaCl, 0.5 mg/mL CaCl₂, 2.5 mg/mL methionine, 1.55 mg/mL L-histidine, 0.4 mg/mL polysorbate 20. The formulation is similar to the formulations described in example 6, yet containing more than 10 times as much methionine.

The formulation was separated into vials and freeze dried using the same freeze drying program (see table 6.1). Prior to freeze drying the vials were spilt into two groups: one group was degassed prior to freeze drying, and the other group was not exposed to degassing.

Degassing procedure: Degassing took place in the freeze dryer prior to the freezing steps as described in example 6.

After freeze drying with and without preceding degassing the freeze dried formulations were reconstituted by 1.1 mL 10 mM histidine, pH 6.0 and analysed for oxidized forms. The percentage of oxidized LC by RP-HPLC was determined right after freeze drying (T=0), and after 1 month (storage of freeze dried formulations) at 30° C. and 40° C., see data in table 7.1

TABLE 7.1
The percentage of oxidized LC in a freeze dried formulation of GP-BDD-
FVIII, with and without preceding degassing.

| | Formulation produced by | Formulation produced by |
|---|---|---|
| | freeze drying with | freeze drying without |
| Storage | preceding degassing | preceding degassing |
| T = 0 | 2.8% oxidized LC | 3.9% oxidized LC |
| 1 month at 30° C. | 3.3% oxidized LC | 5.4% oxidized LC |
| 1 month at 40° C. | 3.3% oxidized LC | 7.2% oxidized LC |

It was observed that oxidation of GP-BDD-FVIII is significantly reduced upon degassing prior to freeze drying. Lower percentage of oxidized LC was detected at T=0 (right after freeze drying), and in particular after storage at 30° and 40° C. during 1 month ((data shown in table 7.1)

Comparison of data in table 6.2 and 7.1 shows that when methionine is increased form 0.22 mg/mL (formulations investigated in example 6) to 2.5 mg/mL (formulation investigated in example 7) the content of oxidized forms is decreased. However, the formulations with highest methionine concentration (2.5 mg/mL, 16.8 mM) still requires degassing to keep FVIII oxidation at acceptable levels (e.g. oxidized forms below 5%).

### Example 8 Methionine Addition

A series of formulations containing glycopegylated B-domain deleted Factor VIII (GP-BDD-FVIII) was prepared to investigate the effect of methionine on LC oxidation. The formulations had the following composition: 1000 IU/mL GP-BDD-FVIII, 70 mg/mL sucrose, 3.5 mg/mL NaCl, 0.5 mg/mL CaCl₂, 1.55 mg/mL L-histidine, 0.4 mg/mL polysorbate 20. The formulations varied with respect to methionine concentration as shown in the results tables 8.1 and 8.2.

The formulations were freeze dried by the freeze drying program shown in table 6.1.

The formulations were freeze dried without preceding degassing.

TABLE 8.1
The percentage of oxidized LC in freeze dried formulations of
GP-BDD-FVIII containing different amounts of methionine.

| | Formulation | | | |
|---|---|---|---|---|
| | #1 | #2 | #3 | #4 |
| Methionine | 0.06 mg/mL | 0.22 mg/mL | 2.5 mg/mL | 10 mg/mL |
| 1 month at | 4.6% | 8.3% | 4.4% | 3.6% (3 weeks |
| 5° C. | | | | at 30° C.) |
| 1 month at | 8.0% | 7.5% | 5.8% | 3.4% (3 weeks |
| 30° C. | | | | at 30° C.) |
| 1 month at | 10.8% | 9.2% | 7.9% | 3.8% (3 weeks |
| 40° C. | | | | at 40° C.) |

It was observed, and shown by data in table 8.1, that oxidation of GP-BDD-FVIII was significantly reduced upon increasing methionine concentration. The lowest levels of oxidized GP-BDD-FVIII were observed for stability samples containing 10 mg/mL methionine.

In addition, it was observed and presented by data in table 8.2 that HMWP % in the investigated formulations of GP-BDD-FVIII was low at all time points (in the accelerated stability study) except from the formulation containing 10 mg/mL methionine. This formulation, #4, had unexpected high HMWP values after freeze drying (at T=0) and after 3 weeks storage. An additional experiment including additional methionine concentrations was made in example 16

TABLE 8.2
The percentage of HMWP in freeze dried formulations of
GP-BDD-FVIII containing different amounts of methionine.

| | Formulation | | | |
|---|---|---|---|---|
| | #1 | #2 | #3 | #4 |
| Methionine | 0.06 mg/mL | 0.22 mg/mL | 2.5 mg/mL | 10 mg/mL |
| Before FD | 0.9% | 0.9% | 0.9% | 0.8% |
| T = 0 | 0.7% | 1.0% | 0.7% | 5.8% |
| 1 month at | 1.0% | 1.0% | 1.0% | |
| 5° C. | | | | |
| 1 month at | 1.0% | 1.0% | 1.0% | 5.6% (3 weeks |
| 30° C. | | | | at 30° C.) |
| 1 month at | 1.0% | 1.0% | 1.5% | 6.1% (3 weeks |
| 40° C. | | | | at 40° C.) |

### Example 9 Decrease in Excipient Concentration to Lower Osmolalityity

Two different formulations were made to investigate the effect on the physical stability of GP-BDD-FVIII when the excipient concentrations were reduced (in order to decrease the osmolality). The composition of excipients in the two formulations (#1 and #2) is described in table 9.1.

TABLE 9.1
Composition of formulation #1 and #2
before freeze drying and estimated osmolality of the formulations
after reconstitution (three times dilution during reconstitution)

| | Formulation #1 | | Formulation #2 | |
|---|---|---|---|---|
| GP-BDD-FVIII | 3000 | U/mL | 3000 | U/mL |
| Sucrose | 12 | mg/mL | 9 | mg/mL |
| NaCl | 36 | mg/mL | 27 | mg/mL |
| CaCl₂ (2H₂O) | 1 | mg/mL | 0.5 | mg/mL |
| Methionine | 0.22 | mg/mL | 0.17 | mg/mL |
| L-histidine | 1.55 | mg/mL | 1.55 | mg/mL |
| polysorbate 20 | 0.4 | mg/mL | 0.3 | mg/mL |
| Estimated osmolality | ~440 | mOsm/kg | ~330 | mOsm/kg |
| After reconstitution (1:3) | | | | |

The two formulations (#1 and #2 described in table 9.1) were freeze dried according to the freeze drying program described in table 9.2.

TABLE 9.2
Freeze drying program

| | | Time | Temp | Alarm | Pressure | R/H |
|---|---|---|---|---|---|---|
| Process step | Step | H:Min | ° C. | Pressure | hPa¹ | step |
| Load | 01 | — | +5 | — | Ambient | Hold |
| Equilibration | 02 | 00:40 | +5 | — | Ambient | Hold |
| Freezing | 03 | 06:00 | −48 | — | Ambient | Ramp |
| Freezing | 04 | 02:00 | −48 | — | Ambient | Hold |
| Annealing | 05 | 01:00 | −30 | — | Ambient | Ramp |
| Annealing | 06 | 10:00 | −30 | — | Ambient | Hold |
| Annealing | 07 | 01:00 | −48 | — | Ambient | Ramp |
| Annealing | 08 | 02:00 | −48 | — | Ambient | Hold |
| Pull vacuum | 09 | 00:01 | −48 | 0.3 | 0.15 | Ramp |
| Primary drying | 10 | 01:00 | −30 | 0.3 | 0.15 | Ramp |
| Primary drying | 11 | 16:00 | −30 | 0.3 | 0.15 | Hold |
| Secondary drying | 12 | 08:00 | +25 | 0.3 | 0.15 | Ramp |
| Secondary drying | 11 | 06:00 | +25 | 0.3 | 0.15 | Hold |

Total time: about 53:40 hours
¹MKS pressure gauge, capacitance manometer gauge

Prior to SE-HPLC analyses (determination of HMWP %), the freeze dried formulations were reconstituted in 1.2 mL histidine buffer pH 6.0 (which was 3× the fill volume of vials prior to freeze drying). The results form SE-HPLC analyses of formulation #1 and #2 (table 9.1) is shown in table 9.3.

Significant increase in HMWP formation (GP-BDD-FVIII aggregation), during storage was observed when the concentration of both NaCl and sucrose was decreased.

TABLE 9.3
Percentage of aggregated GP-BDD-FVIII in two different formulations
(#1 and #2 described in table 9.1) at different time points
in accelerated stability study.

| Stability data | Formulation #1 | Formulation #2 |
|---|---|---|
| HMWP % at T = 0 | 1.2% | 1.5% |
| HMWP after 1 month at 30° C. | 1.5% | 2.0% |
| HMWP after 1 month at 40° C. | 1.9% | 3.7% |

### Example 10 High Salt+Low Sucrose or Low Salt+High Sucrose

A series of different formulations were prepared to investigate the effects of sucrose and NaCl on HMWP formation during freeze drying. The primary variation between the formulations was the concentration of NaCl and sucrose. The composition of formulations is shown in table 10.3.

Due to the variation in NaCl— and sucrose concentration different freeze drying programs were necessarily used to maintain nice appearing freeze drying cakes which were not collapsed during freeze drying. The freeze drying program described in table 10.1 was used for formulation #1, #2, #3 and #4 whereas formulation #5 and #6 were freeze dried using the freeze drying program described in table 10.2.

TABLE 10.1
Freeze drying programme used for Formulation #1, #2, #3 and #4
(described in table 10.3)

| | | Time | Temp | Alarm | Pressure | R/H |
|---|---|---|---|---|---|---|
| Process step | Step | H:Min | ° C. | Pressure | hPa¹ | step |
| Load | 01 | — | +5 | — | Ambient | Hold |
| Equilibration | 02 | 00:40 | +5 | — | Ambient | Hold |
| Freezing | 03 | 06:00 | −48 | — | Ambient | Ramp |
| Freezing | 04 | 02:00 | −48 | — | Ambient | Hold |
| Annealing | 05 | 01:00 | −30 | — | Ambient | Ramp |
| Annealing | 06 | 10:00 | −30 | — | Ambient | Hold |
| Annealing | 07 | 01:00 | −48 | — | Ambient | Ramp |
| Annealing | 08 | 02:00 | −48 | — | Ambient | Hold |
| Pull vacuum | 09 | 00:01 | −48 | 0.3 | 0.15 | Ramp |
| Primary drying | 10 | 01:00 | −30 | 0.3 | 0.15 | Ramp |
| Primary drying | 11 | 16:00 | −30 | 0.3 | 0.15 | Hold |
| Secondary drying | 12 | 08:00 | +25 | 0.3 | 0.15 | Ramp |
| Secondary drying | 11 | 06:00 | +25 | 0.3 | 0.15 | Hold |

meotal time: about 53:40 hours
¹MKS pressure gauge, capacitance manometer gauge

TABLE 10.2
Freeze drying programme used for Formulation #5 and #6
(described in table 10.3)

| | | Time | Temp | Alarm | Pressure | R/H |
|---|---|---|---|---|---|---|
| Process step | Step | H:Min | ° C. | Pressure | hPa¹ | step |
| Load | 01 | — | +5 | — | Ambient | Hold |
| Equilibration | 02 | 01:00 | +5 | — | Ambient | Hold |
| Freezing | 03 | 01:00 | −45 | — | Ambient | Ramp |
| Freezing | 04 | 03:00 | −45 | — | Ambient | Hold |
| Annealing | 05 | 01:00 | −8 | — | Ambient | Ramp |
| Annealing | 06 | 12:00 | −8 | — | Ambient | Hold |
| Annealing | 07 | 01:00 | −45 | — | Ambient | Ramp |
| Annealing | 08 | 03:00 | −45 | — | Ambient | Hold |
| Pull vacuum | 09 | 00:15 | −45 | 0.1 | 0.05 | Ramp |
| Primary drying | 10 | 01:00 | −30 | 0.1 | 0.05 | Ramp |
| Primary drying | 11 | 45:00 | −30 | 0.1 | 0.05 | Hold |
| Secondary | 12 | 10:00 | +48 | 0.1 | 0.05 | Ramp |
| drying | | | | | | |
| Secondary | 11 | 10:00 | +48 | 0.1 | 0.05 | Hold |
| drying | | | | | | |

Total time: about 82:15 hours
¹MKS pressure gauge, capacitance manometer gauge

The formulations were designed to have relative similar osmolality (osmolality<450 mOsm/kg) and similar protein concentration after reconstitution. Thus different reconstitution volumes and protein concentration prior to freeze drying were necessarily used

For formulations with relative high NaCl concentration, formulation #1, #2, #3 and #4 containing 36-30 mg/mL NaCl prior to freeze drying, required 3× dilution during reconstitution (meaning that the reconstitution volume was three times larger than the fill volume in vials prior to freeze drying). For formulations with osmolality<450 mOsm/kg prior to freeze drying (e.g. #5 with 8 mg/mL NaCl and #6 with 3.5 mg/mL NaCl) the reconstitution volume was identical to the fill volume. All formulations were reconstituted by 10 mM histidine buffer pH 6.0.

The percentage of HMWP in the formulations was determined by SE-HPLC before freeze drying, as well as directly after freeze drying. The results are presented in table 10.3.

TABLE 10.3
HMWP percentage and increase in HMWP induced by freeze drying is
shown for six GP−BDD−FVIII formulations (#→#46). The excipient concentration in the table
is defined in mg/mL. The concentration in mg/mL and the osmolality values in the table
relates to the concentration and osmolality after reconstitution

| Formulation | #1 | #2 | #3 | #4 | #5 | #6 |
|---|---|---|---|---|---|---|
| Fill volume: | 1:3 | 1:3 | 1:3 | 1:3 | 1:1 | 1:1 |
| Reconstitution | | | | | | |
| volume | | | | | | |
| GP-BDD-FVIII | 1000 | 1000 | 1000 | 1000 | 1000 | 1000 |
| (IU/mL) | | | | | | |
| Sucrose | 4 | 6 | 6 | 8 | 45 | 70 |
| NaCl | 12 | 12 | 10 | 10 | 8 | 3.50 |
| CaCl2 (2H2O) | 0.33 | 0.33 | 0.33 | 0.33 | 0.50 | 0.50 |
| Methionine | 0.07 | 0.07 | 0.07 | 0.07 | 0.22 | 2.50 |
| L-histidine | 2.07 | 2.07 | 2.07 | 2.07 | 3.10 | 3.10 |
| polysorbate 20 | 0.13 | 0.13 | 0.13 | 0.13 | 0.40 | 0.40 |
| osmolality | 426 | 418 | 364 | 361 | 424 | 372 |
| | mOsm/kg | mOsm/kg | mOsm/kg | mOsm/kg | mOsm/kg | mOsm/kg |
| HMWP (after | 1.2% | 0.9% | 0.7% | 0.6% | 0.7% | 0.9% |
| FD) | | | | | | |
| HMWP (before | 0.4% | 0.4% | 0.4% | 0.4% | 0.5% | 0.7% |
| FD) | | | | | | |

The data in table 10.3 shows relative small variations in HMWP values. This shows that NaCl can be reduced more than 10 times (formulation #1 and #2 contains 36 mg/mL NaCl prior to freeze drying) without compromising the GP-BDD-FVIII stability during freeze drying when the sucrose concentration was increased to 70 mg/mL (formulation #6). In contrast reduction in both NaCl concentration and sucrose concentration was found to reduce the stability of GP-BDD-FVIII during freeze drying and storage (data presented in example 9).

The freeze dried cakes had a nice appearance except from #5 which was collapsed. Additional studies were made investigate stability of freeze dried cake structure at various sucrose and NaCl concentrations (example 19).

The data shows that NaCl concentration can be reduced significantly to decrease osmolality, and thereby allow 1:1 reconstitution, if sucrose is used as protein stabilizer. Note that 1 mg sucrose pr mL water results in much lower increase in the osmolality compared to 1 mg NaCl, which is due its higher molecular weight and the fact that it is not a salt (and thus not dissociated into separate ions each contributing to increased osmolality).

### Example 11 Mannitol Addition

Three different mannitol containing formulations (F9a, F9b, and F9d) were prepared to investigate if mannitol can be used as potential substituent for NaCl (and sucrose). The effect of mannitol on HMWP formation during freeze drying was investigated. GP-BDD-FVIII formulations contained excipients as shown in table 11.3. The formulations contained sucrose and mannitol as main components (in terms of percentage of dry matter) creating the matrix in the freeze dried formulations. The formulation were designed to have an osmolality<400 mOsm/kg after reconstitution.

The formulations were tested with two different freeze drying programs described in table 11.1 and 11.2

TABLE 11.1
Freeze drying program

| | | Time | Temp | Alarm | Pressure | R/H |
|---|---|---|---|---|---|---|
| Process step | Step | H:Min | ° C. | Pressure | hPa¹ | step |
| Load | 01 | — | +5 | — | Ambient | Hold |
| Equilibration | 02 | 01:00 | +5 | — | Ambient | Hold |
| Freezing | 03 | 01:00 | −45 | — | Ambient | Ramp |
| Freezing | 04 | 03:00 | −45 | — | Ambient | Hold |
| Annealing | 05 | 01:00 | −8 | — | Ambient | Ramp |
| Annealing | 06 | 12:00 | −8 | — | Ambient | Hold |
| Annealing | 07 | 01:00 | −45 | — | Ambient | Ramp |
| Annealing | 08 | 03:00 | −45 | — | Ambient | Hold |
| Pull vacuum | 09 | 00:15 | −45 | 0.1 | 0.05 | Ramp |
| Primary drying | 10 | 01:00 | −30 | 0.1 | 0.05 | Ramp |
| Primary drying | 11 | 45:00 | −30 | 0.1 | 0.05 | Hold |
| Secondary | 12 | 10:00 | +48 | 0.1 | 0.05 | Ramp |
| drying | | | | | | |
| Secondary | 11 | 10:00 | +48 | 0.1 | 0.05 | Hold |
| drying | | | | | | |

Total time: about 82:15 hours
¹MKS pressure gauge, capacitance manometer gauge

TABLE 11.2
Freeze drying program

| | | Time | Temp | Alarm | Pressure | R/H |
|---|---|---|---|---|---|---|
| Process step | Step | H:Min | ° C. | Pressure | hPa¹ | step |
| Load | 01 | - | +5 | — | Ambient | Hold |
| Equilibration | 02 | 01:00 | +5 | — | Ambient | Hold |
| Freezing | 03 | 01:00 | −45 | — | Ambient | Ramp |
| Freezing | 04 | 03:00 | −45 | — | Ambient | Hold |
| Annealing | 05 | 01:00 | −20 | — | Ambient | Ramp |
| Annealing | 06 | 05:00 | −20 | — | Ambient | Hold |
| Annealing | 07 | 01:00 | −45 | — | Ambient | Ramp |
| Annealing | 08 | 03:00 | −45 | — | Ambient | Hold |
| Pull vacuum | 09 | 00:15 | −45 | 0.3 | 0.15 | Ramp |
| Primary drying | 10 | 01:00 | −25 | 0.3 | 0.15 | Ramp |
| Primary drying | 11 | 25:00 | −25 | 0.3 | 0.15 | Hold |
| Secondary drying | 12 | 10:00 | +48 | 0.3 | 0.15 | Ramp |
| Secondary drying | 11 | 06:00 | +48 | 0.3 | 0.15 | Hold |

Total time: about 57 hours
¹MKS pressure gauge, capacitance manometer gauge

The HMWP data (in table 11.4) shows that, for both tested freeze drying programs (table 11.1 and 11.2), the mannitol containing formulations had higher percentage of HMWP compared to formulations without mannitol (see data presented in table 10.3).

It was found that for the mannitol containing formulations (F9a, F9b, F9d, in table 11.3) high sucrose (20 mg/mL sucrose+20 mg/mL mannitol), compared to low sucrose (10 mg/mL sucrose+25 mg/mL mannitol), results in lower percentage of HMWP This is in agreement with observations in example 10 showing that sucrose is a stabilizing excipient during freeze drying.

TABLE 11.3
Three different mannitol containing GP-BDD-FVIII formulations (#F9a,
b, and d) were prepared and presented in this example F9a, F9b and F9d.

| | Formulation | | |
|---|---|---|---|
| | F9a | F9b | F9d |
| GP-BDD-FVIII (U/mL) | 1000 | 2000 | 2000 |
| Sucrose (mg/mL) | 10 | 10 | 20 |
| NaCl (mg/mL) | 3.5 | 3.5 | 3.5 |
| Mannitol (mg/mL) | 25 | 25 | 20 |
| CaCl₂ (2H₂O) (mg/mL) | 0.5 | 0.5 | 0.5 |
| Methionine (mg/mL) | 2.5 | 2.5 | 2.5 |
| L-histidine (mg/mL) | 1.55 | 1.55 | 1.55 |
| polysorbate 20 (mg/mL) | 0.4 | 0.4 | 0.4 |
| Estimated osmolality | 333 | 333 | 335 |
| (mOsm) | | | |

TABLE 11.4
HMWP % in formulations 9a, 9b, and 9d quantified by SE-HPLC before
freeze drying (FD) and after FD, and after storage 1 month at 40 C.

| | Formulation | | |
|---|---|---|---|
| | F9a | F9b | F9d |
| HMWP before FD | 0.8% | 1.3% | 1.1% |
| HMWP after FD (FD program 11.1) | 2.1% | 2.3% | 1.6% |
| HMWP after 1 month at 40 C. (FD program 11.1) | 2.8% | 3.4% | 1.8% |
| HMWP after FD (FD program 11.2) | 5.6% | 4.7% | 2.8% |
| HMWP after 1 month at 40 C. (FD program 11.2) | 6.7% | 5.3% | 3.3% |

### Example 12 Stabilizing Effect of Trehalose on GP-BDD-FVIII

Four formulations were prepared to investigate potential stabilizing effects of trehalose (formulation #63, 64, 65, 66). The formulations contained 0.5 mg/mL GP-BDD-FVIII (about 5000 U/mL), 3.5 mg/mL NaCl, 2 mg/mL CaCl₂*2H₂O, 1.6 mg/mL histidine, 2.5 mg/mL methionine, 15 mg/mL sucrose, 0.2 mg/mL tween 20, pH 7. The formulations differed with regards to the trehalose concentration which were: 60 mg/mL, 80 mg/mL, 90 mg/mL and 100 mg/mL.

These formulations were not freeze dried, but stressed as liquid samples at 50° C. during 30 minutes to compare the physical stability of GP-BDD-FVIII during heat exposure. The samples were analysed by SE-HPLC for quantification of HMWP % before and after heat exposure. The data is shown in table 12.1.

TABLE 12.1
HMWP % for four different trehalose containing formulations

| | Formulation | Formulation | Formulation | Formulation |
|---|---|---|---|---|
| | #63 with | #64 with | #65 with | with #66 |
| | 60 mg/mL | 80 mg/mL | 90 mg/mL | 100 mg/mL |
| | trehalose | trehalose | trehalose | trehalose |
| HMWP % | *Not | 1.8% | 1.8% | 1.8% |
| before stress | analysed | | | |
| HMWP % after | 7.9% | 6.5% | 4.8% | 4.2% |
| 30 min at | | | | |
| 50° C. | | | | |

*HMWP was not quantified due to chromatographic issues during analyses of this sample

A clear stabilizing effect of trehalose was observed as lower HMWP % in stressed samples (upon increased concentration of trehalose).

### Example 13 Stabilizing Effect of Histidine on GP-BDD-FVIII

Three formulations were prepared to investigate potential stabilizing effects of histidine (formulation #54, 55, 56). The formulations contained 0.5 mg/mL GP-BDD-FVIII (about 5000 U/mL), 3.5 mg/mL NaCl, 2 mg/mL CaCl₂*2H₂O, 2.5 mg/mL methionine, 70 mg/mL sucrose, 0.2 mg/mL tween 20, pH 7. The formulations differed with regards to the histidine concentration which was: 3 mg/mL, 5 mg/mL and 7 mg/mL.

These formulations were not freeze dried, but stressed as liquid samples at 50° C. during 30 minutes to compare the physical stability of GP-BDD-FVIII during heat exposure. The samples were analysed by SE-HPLC for quantification of HMWP % before and after heat exposure. The data is shown in table 13.1.

TABLE 13.1
HMWP % for four different histidine containing formulations

| | | | Formulation |
|---|---|---|---|
| | Formulation #54 | Formulation #55 | #56 |
| | with 3 mg/mL | with 5 mg/mL | with 7 mg/mL |
| | histidine | histidine | histidine |
| HMWP % before | 1.9% | 1.8% | 1.8% |
| stress | | | |
| HMWP % after | 7.4% | 5.8% | 4.2% |
| 30 min at 50° C. | | | |

A clear stabilizing effect of histidine was observed as lower HMWP % in stressed samples (upon increased concentration of histidine). In contrast, no effects of arginine, glutamine and succinate were observed in similar formulations exposed to same stress conditions.

### Example 14 Effect of Two Different Degassing Procedures

The influence of the degassing procedure on the oxidation of GP-BDD-FVIII was investigated applying a degassing time of 60 minutes and with/without equilibration to atmospheric pressure with nitrogen before the start of the freeze drying process. All formulations were freeze dried according to the program described in table 25.1.

The GP-BDD-FVIII formulation contains: 2000 IU/mL GP-BDD-FVIII, 3.5 mg/mL NaCl, 0.5 mg/mL CaCl₂, 1.55 mg/mL L-histidine, 2.5 mg/mL methionine, 70 mg/mL sucrose, and 0.4 mg/mL polysorbate 20.
Vials with liquid formulation were placed on the shelf of the freeze dryer and the shelf was cooled to 5° C. The formulations were degassed in two separate freeze dryers prior to the freezing step according to the following procedures:
Freeze dryer 1: Oxygen was removed from the liquid by applying low pressure (100 mbar) during 60 minutes at ±20° C. The pressure was equilibrated to 1 atm (1013 mbar) by nitrogen gas. The degassing procedure was only performed once before the freezing step.
Freeze dryer 2: Oxygen was removed from the liquid by applying low pressure (100 mbar) during 60 minutes at ±20° C. The degassing procedure was only performed once before the freezing step. The freezing step was started immediately after the degassing procedure without increasing the pressure to atmospheric pressure.

TABLE 14.1
The percentage of oxidized protein in freeze dried formulations of
GP-BDD-FVIII degassed for 1 × 60 minutes with/without
equilibration of pressure to 1 atm (1013 mbar).

| | Content of Oxidised forms (%) | |
|---|---|---|
| | With | |
| Sample for RP-HPLC analysis | equalibration | Without equalibration |
| Drug substance before | 2.3 | 2.3 |
| formulation to Drug product | | |
| Before freeze drying | 2.3 | 2.3 |
| After freeze drying | 2.5 | 2.6 |
| T = 0 | | |
| 3 weeks storage of freeze dried | 4.4 | 4.5 |
| vials at +40° C. | | |

Data show that using a degassing period of 60 minutes gives a higher content of oxidised forms compared to a degassing period of 2×20 minutes. Furthermore the data shows that it is of no importance with respect to the content of oxidised forms whether an equilibration to atmospheric pressure is performed.

### Example 15 Sc Formulations with Glyco-HEPylated-BDD-FVIII

In one experiment various strengths of various long acting FVIII molecules were freeze dried and studied. The investigated long acting FVIII proteins were: GP-BDD-FVIII and a HEPylated version of the same BDD FVIII molecule (glyco-Hepylated B-domain depleted FVIII, GH-BDD-FVIII).

Unlike in the previous examples, the protein in this example were purified and stored in a high salt buffer prior to the formulation work: 12 mg/mL sucrose, 36 mg/mL NaCl, 1 mg/mL CaCl₂, 6 mg/mL L-histidine, 0.22 mg/mL methionine, 0.4 mg/mL Tween 80. The proteins were buffer-exchanged into a buffer containing: 70 mg/mL sucrose, 3.5 mg/mL NaCl, 0.5 mg/mL CaCl₂, 1.55 mg/mL L-histidine, 2.5 mg/mL methionine, 0.4 mg/mL polysorbate 20. The proteins were concentrated to about 9000 IU/mL (stock solutions). The various strengths (250 IU/mL, 2000 IU/mL, 6000 IU/mL) of the FVIII molecules were prepared by dilution of the 9000 IU/mL stock solutions.

All protein solutions were freeze dried according to the program described in table 15.1. The formulations were degassed in the freeze dryer prior to the freezing step according to following procedure: Oxygen was removed from the liquid by applying low pressure (100 mbar) during 20 minutes at +20° C. The pressure was equilibrated to 1 atm (1013 mbar) by nitrogen gas. This procedure was repeated twice before the freezing step.

TABLE 15.1
Freeze drying program

| | | Time | | Alarm | Pressure | R/H |
|---|---|---|---|---|---|---|
| Process step | Step | H:Min | ° C. | Pressure | hPa¹ | step |
| Load | 01 | — | +5 | — | Ambient | Hold |
| Equilibration | 02 | 01:00 | +5 | — | Ambient | Hold |
| Freezing | 03 | 01:00 | −45 | — | Ambient | Ramp |
| Freezing | 04 | 03:00 | −45 | — | Ambient | Hold |
| Annealing | 05 | 01:00 | −8 | — | Ambient | Ramp |
| Annealing | 06 | 12:00 | −8 | — | Ambient | Hold |
| Annealing | 07 | 01:00 | −45 | — | Ambient | Ramp |
| Annealing | 08 | 03:00 | −45 | — | Ambient | Hold |
| Pull vacuum | 09 | 00:15 | −45 | 0.1 | 0.05 | Ramp |
| Primary drying | 10 | 01:00 | −30 | 0.1 | 0.05 | Ramp |
| Primary drying | 11 | 45:00 | −30 | 0.1 | 0.05 | Hold |
| Secondary drying | 12 | 14:00 | +40 | 0.1 | 0.05 | Ramp |
| Secondary drying | 11 | 12:00 | +40 | 0.1 | .05 | Hold |

Total time: about 94 hours
¹MKS pressure gauge, capacitance manometer gauge

The vials were filled with 1 mL formulation prior to freeze drying. After freeze drying the freeze dried formulations were reconstituted by 1 mL 10 mM histidine buffer and analysed by SE-HPLC to quantify the content of aggregated protein (HMWP %). These data is shown in table 15.2.

TABLE 15.2
The content of protein aggregates, shown as HMWP %, in
formulations of GP-BDD-FVIII and GH-BDD-FVIII before
freeze drying and after freeze drying.

| | HMWP % determined by SE-HPLC | | | | | |
|---|---|---|---|---|---|---|
| | GP-BDD-FVIII | | | GH-BDD-FVIII | | |
| | 250 | 2000 | 6000 | 250 | 2000 | 6000 |
| | IU/ | IU/ | IU/ | IU/ | IU/ | IU/ |
| | vial | vial | vial | vial | vial | vial |
| Before freeze drying | 1.6% | 2.3% | 2.9% | 2.2% | 1.7% | 2.3% |
| After freeze drying | 2.1% | 3.2% | 4.6% | 1.5% | 1.8% | 3.3% |
| and 4 weeks storage | | | | | | |
| of freeze dried vials | | | | | | |
| at −80° C. | | | | | | |

It was observed that different long acting FVIII molecules (GP-BDD-FVIII and GH-BDD-FVIII) can be formulated and freeze dried into a (sc) formulation containing high strength (6000 IU/mL), high concentration of sucrose, and low osmolality (350-400 mOsm/kg, before freeze drying and after reconstitution).

### Example 16 Degassing, Methionine Addition and Reduction of FVIII LC Oxidation

In one experiment freeze dried formulations containing 1000 IU/mL GP-BDD-FVIII, 70 mg/mL sucrose, 3.5 mg/mL NaCl, 0.5 mg/mL CaCl₂, 1.55 mg/mL L-histidine, 0.4 mg/mL polysorbate 20 were prepared with various concentrations of methionine: 0.25, 0.5, 1, 2.5, 5, 7.5, 10 mg/mL.

Formulations were filled into freeze drying vials, with a fill volume of 1 mL. The formulations were freeze dried according to the program described in table 15.1. All formulations were both freeze dried with- and without preceding degassing. Degassing took place in the freeze dryer prior to the freezing step according to the procedure described in example 15.

After freeze drying the vials were stored at three different temperatures: −80 C, +30 C and +40 C, for storage 1 month. After storage the freeze dried formulations were reconstituted by 1 mL histidine buffer pH 6.0 and analysed directly by SE-HPLC to quantify the content of aggregated protein (HMWP %). Aliquots of reconstituted formulations were stored at −80 C until RP-HPLC analysis for quantification of protein oxidation (oxidized forms %). Data is shown in table 16.1 (HMWP %), 16.2 (oxidized forms % in formulations which were not degassed prior to freeze drying) and 16.3 (oxidized forms % in degassed formulations).

TABLE 16.1
Content of protein aggregates, shown as HMWP %, in formulations of
GP-BDD-FVIII before freeze drying and after freeze drying. HMWP %
is shown for six formulations with various methionine concentrations,
stored at different temperatures after freeze drying.

| | HMWP % (Methionine) | | | | | |
|---|---|---|---|---|---|---|
| | 0.25 | 1 | 2.5 | 5 | 7.5 | 10 |
| | mg/ | mg/ | mg/ | mg/ | mg/ | mg/ |
| | mL | mL | mL | mL | mL | mL |
| Before freeze drying | 1.5% | 1.5% | 1.5% | 1.5% | 1.5% | 1.4% |
| 1 month at −80° C. | 1.6% | 1.5% | 1.6% | 1.6% | 1.7% | 2.4% |
| (degassing before freeze | | | | | | |
| drying) | | | | | | |
| 1 month at −80° C. (freeze | 1.7% | 1.6% | 1.6% | 1.7% | 1.8% | 2.1% |
| drying without preceding | | | | | | |
| degassing) | | | | | | |
| 1 month at +30° C. | 1.8% | 1.7% | 1.7% | 1.9% | 2.0% | 2.4% |
| (degassing before freeze | | | | | | |
| drying) | | | | | | |
| 1 month at +30° C. (freeze | 1.8% | 1.7% | 1.7% | 1.8% | 1.9% | 2.2% |
| drying without preceding | | | | | | |
| degassing) | | | | | | |
| 1 month at +40° C. | 1.8% | 1.7% | 1.8% | 2.0% | 2.3% | 2.5% |
| (degassing before freeze | | | | | | |
| drying) | | | | | | |
| 1 month at +40° C. (freeze | 1.9% | 1.7% | 1.8% | 1.9% | 2.2% | 2.4% |
| drying without preceding | | | | | | |
| degassing) | | | | | | |

Low percentage of aggregated protein (HMWP %<3%) was observed in the entire investigated methionine concentration range, both before and after freeze drying. However, after freeze drying the highest amount of HMWP was observed in formulations containing the highest methionine concentrations. The data shows that the optimal methionine concentration with regards to limiting HMWP formation is between 0.25-7.5 mg/mL. The data further show that degassing prior to freeze drying has no influence on GP-BDD-FVIII aggregation.

TABLE 16.2
Content of oxidized protein, shown as oxidized forms %, in formulations
of GP-BDD-FVIII before freeze drying and after freeze drying. No
degassing was applied prior to freeze drying

| | Ox. forms % (Methionine) | | | | | |
|---|---|---|---|---|---|---|
| | 0.25 | 1 | 2.5 | 5 | 7.5 | 10 |
| | mg/ | mg/ | mg/ | mg/ | mg/ | mg/ |
| | mL | mL | mL | mL | mL | mL |
| Before freeze drying | 2.8% | 2.8% | 2.7% | 2.7% | 2.7% | 2.8% |
| 1 month at −80° C. (freeze | 3.5% | 3.3% | 3.1% | 2.9% | 3% | 2.9% |
| drying without preceding | | | | | | |
| degassing) | | | | | | |
| 1 month at +30° C. (freeze | 5.3% | 5.6% | 5.0% | 4.1% | 3.9% | 3.4% |
| drying without preceding | | | | | | |
| degassing) | | | | | | |
| 1 month at +40° C. (freeze | 7.0% | 6.2% | 6.4% | 4.8% | 4.4% | 4.2% |
| drying without preceding | | | | | | |
| degassing) | | | | | | |

The data in table 16.2 shows that FVIII oxidation (oxidized forms %) after freeze drying is increased when the storage temperature is increased (vertical comparison of data), and that this oxidation is decreased when the concentration of methionine is increased (horizontal comparison of data).

Upon storage at 30 C and 40 C, degassing prior to freeze drying is essential to reduce GP-BDD-FVIII oxidation (especially when the methionine concentration is low). Comparison of data in table 16.2 (formulations which were not degassed prior to freeze drying) with data in table 16.3 (degassed formulations) it is clear that degassing decreases FVIII oxidation. Oxidation of GP-BDD-FVIII primarily occurs during storage at elevated temperature, but is also observed during freeze drying or at −80 C if the formulations are not degassed and contain less than 5 mg/mL methionine.

The data shows that degassing and methionine are essential to the storage stability of the investigated freeze dried formulations.

One data point in these series of data can be questioned. The content of oxidized forms in the formulation containing 10 mg/mL methionine (degassed before freeze drying and stored 1 month at 30 C) is higher than expected and it is plausible that a handling error has been made e.g. during labelling.

TABLE 16.3
Content of oxidized protein, shown as oxidized forms %, in formulations
of GP-BDD-FVIII before freeze drying and after freeze drying. Freeze
drying included degassing of samples

| | Ox. forms % (Methionine variation) | | | | | |
|---|---|---|---|---|---|---|
| | 0.25 | 1 | 2.5 | 5 | 7.5 | 10 |
| | mg/ | mg/ | mg/ | mg/ | mg/ | mg/ |
| | mL | mL | mL | mL | mL | mL |
| Before freeze drying | 2.8% | 2.8% | 2.7% | 2.7% | 2.7% | 2.8% |
| 1 month at −80° C. | 2.9% | 3.0% | 2.8% | 2.8% | 2.9% | 2.8% |
| (degassing before freeze | | | | | | |
| drying) | | | | | | |
| 1 month at +30° C. | 4.1% | 3.3% | 3.3% | 3.2% | 3.1% | (4.2%) |
| (degassing before freeze | | | | | | |
| drying) | | | | | | |
| 1 month at +40° C. | 5.1% | 4.2% | 4.0% | 3.8% | 4% | 3.2% |
| (degassing before freeze | | | | | | |
| drying) | | | | | | |

### Example 17 Stabilizing Effect of CaCl₂

In one experiment freeze dried formulations containing GP-BDD-FVIII, 70 mg/mL sucrose, 3.5 mg/mL NaCl, 2.5 mg/mL methionine, 1.55 mg/mL L-histidine, 0.4 mg/mL polysorbate 20 were prepared with various concentrations of CaCl₂: 1.2 mM, 2.3 mM, 4.1 mM, 6.8 mM, 12.2 mM (0.17 mg/mL, 0.34 mg/mL, 0.6 mg/mL, 1 mg/mL and 1.8 mg/mL CaCl2*2H2O). Formulations contained either 0.11 mg/mL or 0.17 mg/mL GP-BDD-FVIII (indicated by data table 17.1)

Formulations were filled into freeze drying vials, with a fill volume of 1 mL. The formulations were freeze dried according to the program described in table 15.1. All formulations were exposed to preceding degassing. Degassing took place in the freeze dryer prior to the freezing step according to the procedure described in example 15.

After freeze drying the vials were stored at three different temperatures: −80° C., +30° C. and +40° C., for storage 1 month. After storage the freeze dried formulations were reconstituted by 1 mL histidine buffer pH 6.0 and analysed directly by SE-HPLC to quantify the content of aggregated protein (HMWP %).

TABLE 17.1
Content of protein aggregates (HMWP %), before and after freeze drying,
in GP-BDD-FVIII formulations. HMWP % is shown for five formulations
with various CaCl₂ concentrations. The freeze dried formulations were
stored at different temperatures

| | | | | | | |
|---|---|---|---|---|---|---|
| HMWP % | CaCl₂ | 1.2 mM | 2.3 mM | 4.1 mM | 6.8 mM | 12.2 |
| | concentration: | | | | | mM |
| | GP-BDD- | 0.11 | 0.11 | 0.17 | 0.17 | 0.17 |
| | FVIII | mg/ | mg/ | mg/ | mg/ | mg/ |
| | concentration: | mL | mL | mL | mL | mL |

| | | | | | |
|---|---|---|---|---|---|
| Before freeze drying | 2.14% | 2.09% | 1.91% | 1.77% | 1.63% |
| 1 month storage of freeze | 2.60% | 2.37% | 2.02% | 1.78% | 1.63% |
| dried vials at −80° C. | | | | | |
| 1 month storage of freeze | 2.73% | 2.42% | 2.05% | 1.79% | 1.63% |
| dried vials at +30° C. | | | | | |
| 1 month storage of freeze | 2.80% | 2.48% | 2.09% | 1.78% | 1.64% |
| dried vials at +40° C. | | | | | |

Increase in CaCl₂ stabilizes GP-BDD-FVIII during freeze drying and during storage of freeze dried GP-BDD-FVIII formulations CaCl₂ was increased from 1.2 to 12.2 mM in this study, and data shows that in formulations with 6.8 mM CaCl₂ (1 mg/mL CaCl₂*2H₂O), or higher GP-BDD-FVIII aggregation is unaffected by freeze drying and 1 month storage at elevated temperatures

### Example 18 Variation in Tween 20 Concentration

In one experiment freeze dried formulations containing 2000 IU/mL GP-BDD-FVIII, 70 mg/mL sucrose, 3.5 mg/mL NaCl, 2.5 mg/mL methionine, 1.55 mg/mL L-histidine, 3.4 mM CaCl₂ were prepared with various concentrations of tween 20 (polysorbate 20): 0.1 mg/mL, 0.2 mg/mL 0.3 mg/mL and 0.4 mg/mL.

Formulations were filled into freeze drying vials, with a fill volume of 1 mL. The formulations were degassed prior to freeze drying according to procedure described in example 15. The formulations were freeze dried according to the program described in table 15.1.

After freeze drying the vials were stored at three different temperatures: −80° C., +30° C. and +40° C., for storage 1 month. After storage the freeze dried formulations were reconstituted by 1 mL histidine buffer pH 6.0 and analysed directly by SE-HPLC to quantify the protein concentration (GP-BDD-FVIII in mg/mL) and the content of aggregated protein (HMWP %). Aliquots of reconstituted formulations were stored at −80 C until quantification of protein oxidation (oxidized forms %) by RP-HPLC analysis. Data is shown in table 18.1 (HMWP %), 18.2 (protein concentration) and 18.3 (oxidized forms %).

TABLE 18.1
HMWP % in formulations of GP-BDD-FVIII. HMWP % is shown for
four formulations (Tween 20 variation) before - and after freeze drying,
as well as after 1 month storage at different temperatures.

| | HMWP % | | | |
|---|---|---|---|---|
| | 0.1 | 0.2 | 0.3 | 0.4 |
| | mg/mL | mg/mL | mg/mL | mg/mL |
| | Tween 20 | Tween 20 | Tween 20 | Tween 20 |
| Before freeze drying | 2.3% | 2.5% | 2.2% | 2.1% |
| 1 month storage of freeze | 2.2% | 2.3% | 2.3% | 2.4% |
| dried vials at −80° C. | | | | |
| 1 month storage of freeze | 2.4% | 2.4% | 2.5% | 2.5% |
| dried vials at +30° C. | | | | |
| 1 month storage of freeze | 2.5% | 2.5% | 2.6% | 2.6% |
| dried vials at +40° C. | | | | |

TABLE 18.2
Concentration of GP-BDD-FVIII before freeze drying and after freeze drying and
reconstitution. Formulations contained four different Tween 20 concentrations and were
stored 1 month at three different temperatures as indicated

| | [GP-BDD-FVIII] | | | |
|---|---|---|---|---|
| | 0.1 mg/mL | 0.2 mg/mL | 0.3 mg/mL | 0.4 mg/mL |
| | Tween 20 | Tween 20 | Tween 20 | Tween 20 |
| Before freeze drying | 0.22 mg/mL | 0.23 mg/mL | 0.23 mg/mL | 0.23 mg/mL |
| 1 month storage of freeze dried | 0.22 mg/mL | 0.22 mg/mL | 0.22 mg/mL | 0.22 mg/mL |
| vials at −80° C. | | | | |
| 1 month storage of freeze dried | 0.22 mg/mL | 0.22 mg/mL | 0.22 mg/mL | 0.22 mg/mL |
| vials at +30° C. | | | | |
| 1 month storage of freeze dried | 0.22 mg/mL | 0.22 mg/mL | 0.22 mg/mL | 0.22 mg/mL |
| vials at +40° C. | | | | |

TABLE 18.3
The percentage of oxidized protein in freeze dried formulations
of GP-BDD-FVIII containing various concentrations of Tween 20

| | Oxidized forms % | | | |
|---|---|---|---|---|
| | 0.1 | 0.2 | 0.3 | 0.4 |
| | mg/mL | mg/mL | mg/mL | mg/mL |
| | Tween 20 | Tween 20 | Tween 20 | Tween 20 |
| Before freeze drying | 2.4% | 2.3% | 2.4% | 2.4% |
| 1 month storage of freeze | 2.5% | 2.5% | 2.5% | 2.5% |
| dried vials at −80° C. | | | | |
| 1 month storage of freeze | 3.1% | 3.1% | 3.0% | 3.0% |
| dried vials at +30° C. | | | | |
| 1 month storage of freeze | 3.0% | 3.0% | 3.2% | 2.9% |
| dried vials at +40° C. | | | | |

HMWP, protein concentration and protein oxidation is not affected by changes in Tween 20 concentration within the investigated concentration range of 0.1-0.4 mg/mL.

### Example 19 Effects of NaCl and Sucrose Ratio on Freeze Dried Cake Structure

In one experiment freeze dried placebo formulation containing 2.5 mg/mL methionine, 1.55 mg/mL L-histidine, 0.4 mg/mL polysorbate 20 was prepared with various concentrations of CaCl₂, NaCl and sucrose as indicated by tables below. The formulations were made to investigate the appearance of freeze dried cakes.

Formulations were filled into freeze drying vials, with a fill volume of 1 mL. The formulations were freeze dried according to the program described in table 15.1. No preceding degassing steps were performed.

After freeze drying the vials were stored at 5° C. The vials were visually inspected after freeze drying and after storage at 2 years, to evaluate the appearance of the freeze drying cakes. Results are presented in table 19.1 and 19.2.

TABLE 19.1
Visual appearance of freeze dried placebo formulations after 2 years
storage at room temperature is shown. A nice appearing cake
structure is indicated by “+”, a collapsed, or partly collapsed, cake
structure is indicated by “−” and formulations not tested is
indicated by “NT”.

| | | NaCl (mg/mL) | | | | | | | | |
|---|---|---|---|---|---|---|---|---|---|---|
| | | 2.5 | 2.9 | 3.5 | 4 | 4.5 | 5 | 5.5 | 6 | 6.5 |
| CaCl2 0.5 | mg/mL, | + | + | + | + | − | − | − | − | − |
| Sucrose 48 | mg/mL | | | | | | | | | |
| CaCl2 0.75 | mg/mL, | NT | NT | + | + | − | NT | NT | NT | NT |
| Sucrose 48 | mg/mL | | | | | | | | | |
| CaCl2 0.5 | mg/mL, | + | + | + | + | + | + | + | − | − |
| Sucrose 58 | mg/mL | | | | | | | | | |
| CaCl2 0.75 | mg/mL, | NT | NT | + | + | + | NT | NT | NT | NT |
| Sucrose 58 | mg/mL | | | | | | | | | |
| CaCl2 0.5 | mg/mL, | + | + | + | + | + | + | + | + | + |
| Sucrose 68 | mg/mL | | | | | | | | | |
| CaCl2 0.75 | mg/mL, | NT | NT | + | + | − | NT | NT | NT | NT |
| Sucrose 68 | mg/mL | | | | | | | | | |
| CaCl2 0.5 | mg/mL, | + | + | + | + | + | + | + | − | + |
| Sucrose 78 | mg/mL | | | | | | | | | |
| CaCl2 0.75 | mg/mL, | NT | NT | + | + | + | NT | NT | NT | NT |
| Sucrose 78 | mg/mL | | | | | | | | | |
| CaCl2 0.5 | mg/mL, | + | + | + | + | + | + | + | + | + |
| Sucrose 88 | mg/mL | | | | | | | | | |
| CaCl2 0.75 | mg/mL, | NT | NT | + | + | + | NT | NT | NT | NT |
| Sucrose 88 | mg/mL | | | | | | | | | |
| CaCl2 0.5 | mg/mL, | + | + | + | + | + | + | + | + | + |
| Sucrose 98 | mg/mL | | | | | | | | | |
| CaCl2 0.75 | mg/mL, | NT | NT | + | + | + | NT | NT | NT | NT |
| Sucrose 98 | mg/mL | | | | | | | | | |
| CaCl2 0.5 | mg/mL, | + | + | + | + | + | + | + | + | + |
| Sucrose 108 | mg/mL | | | | | | | | | |
| CaCl2 0.75 | mg/mL, | NT | NT | + | + | + | NT | NT | NT | NT |
| Sucrose 108 | mg/mL | | | | | | | | | |
| CaCl2 0.5 | mg/mL, | + | + | + | + | + | + | + | + | + |
| Sucrose 116 | mg/mL | | | | | | | | | |
| CaCl2 0.75 | mg/mL, | NT | NT | + | + | + | NT | NT | NT | NT |
| Sucrose 116 | mg/mL | | | | | | | | | |

Visual investigation of vials after freeze drying and storage shows that increasing concentration of sucrose 68-116 mg/mL increases the number of nice appearing freeze dried cakes (independent on NaCl and CaCl₂). At lower sucrose concentrations 48-58 mg/mL FD (freeze dried) cake collapse is observed when NaCl concentrations are “high” 4.5-6.5 mg/mL (results presented in table 19.1). Thus a combination of NaCl>5.5 mg/mL and “low” sucrose concentration<58 mg/mL (table 19.1) does not provide stable a FD cake structure (does not provide nice appearing FD cakes).

Increasing sucrose/NaCl ratio improves the FD cake structure. No collapsed FD cakes were observed for formulations containing: 88 mg/mL sucrose without NaCl, 81 mg/mL sucrose and 0.75 mg/mL NaCl, 73 mg/mL sucrose and 1.5 mg/mL NaCl, 68 mg/mL sucrose and 2.5 mg/mL NaCl. No effect on visual appearance of CaCl₂ in the range of: 3.4-13.6 mM (0.5-2 mg/mL CaCl₂*2H₂O) was observed (table 19.2)

TABLE 19.2
Visual appearance of freeze dried placebo formulations after 2 years
storage at room temperature is shown. A nice appearing cake
structure is indicated by “+” and a collapsed, or partly collapsed,
cake structure is indicated by “−”

| | Sucrose/NaCl (mg/mL) | | | | | | | | | |
|---|---|---|---|---|---|---|---|---|---|---|
| | 88/ | 81/ | 73/ | 68/ | 63/ | 58/ | 53/ | 48/ | 43/ | 38/ |
| | 0 | 0.75 | 1.5 | 2 | 2.5 | 3 | 3.5 | 4 | 4.5 | 6 |
| CaCl₂ (2•H₂O) | + | + | + | + | + | + | + | − | − | − |
| 0.5 mg/mL | | | | | | | | | | |
| CaCl₂ (2•H₂O) | + | + | + | + | + | + | + | − | − | − |
| 0.75 mg/mL | | | | | | | | | | |
| CaCl₂ (2•H₂O) | + | + | + | + | + | − | + | − | − | − |
| 1 mg/mL | | | | | | | | | | |
| CaCl₂ (2•H₂O) | + | + | + | + | + | + | + | − | − | − |
| 1.5 mg/mL | | | | | | | | | | |
| CaCl₂ (2•H₂O) | + | + | + | + | + | + | + | − | − | − |
| 2 mg/mL | | | | | | | | | | |

### Example 20 GP-BDD-FVIII Formulation and Stability

In one experiment freeze dried formulations containing 0.2 mg/mL and 0.4 mg/mL GP-BDD-FVIII were prepared. The excipient content was: 70 mg/mL sucrose, 3.5 mg/mL NaCl, 2.5 mg/mL methionine, 1.55 mg/mL L-histidine, 3.4 mM CaCl₂, 0.4 mg/mL Tween 20. Formulations were filled into freeze drying vials, with a fill volume of 1 mL. The formulations were freeze dried according to the program described in table 15.1. All formulations were degassed prior to freeze drying. Degassing took place in the freeze dryer prior to the freezing step according to the procedure described in example 15.

After freeze drying the vials were stored at three different temperatures: +5 C, +30 C and +40 C, in order to investigate the stability during several months. After the intended storage period (1 month, 3 months or 6 months) the freeze dried formulations were reconstituted by 1 mL histidine buffer pH 6.0 and analysed directly. Three different analytical methods were included: SE-HPLC for quantifications of protein aggregation (HMWP %), RP-HPLC for quantification of protein oxidation/FVIII LC oxidation (oxidized forms %) and chromogenic FVIII assay to quantify the FVIII activity in the formulations. In this assay FVIII functions as a cofactor in the activation of factor X in the presence of Factor IXa, Ca2+ and phospholipid. Factor Xa hydrolyses the chromogenic substrate (S-2765), and the chromophore group, pNA, is released and the absorbance at 415 nm is measured on a plate reader. The amount of factor Xa and the formed pNA is proportional to the content of factor VIII in the analysed sample. This linear correlation is used to establish the content of active factor VIII in the sample by comparison with a reference which is analysed in parallel.

These analyses were performed before freeze drying, after freeze drying, and after storage as indicated by data tables below: Table 20.1 (FVIII activity), table 20.2 (HMWP %) table 20.3 (oxidized forms %).

TABLE 20.1
Analysis of Bioactivity in formulation of GP-BDD-FVIII with
concentration of 0.2 mg/ml. The analysis was performed after
freeze drying (at t = 0) and after 1, 3 and 6 months storage at +30 C.

| | GP-BDD-FVIII |
|---|---|
| Bioactivity | 0.2 mg/mL |
| T = 0 | 2250 IU/ml |
| 1 month storage of freeze dried vials at +30° C. | 2086 IU/ml |
| 3 month storage of freeze dried vials at +30° C. | 2297 IU/ml |
| 6 month storage of freeze dried vials at +30° C. | 2280 IU/ml |

The investigated formulation provides fully active GP-BDD-FVIII for at least 6 months storage at 30° C.

TABLE 20.2
HMWP %, in formulations of GP-BDD-FVIII with two different
concentrations. The analysis was performed before freeze drying,
after freeze drying (at t = 0), and after 1 month storage
at three different temperatures.

| | HMWP % | |
|---|---|---|
| | GP-BDD-FVIII | GP-BDD-FVIII |
| | 0.2 mg/mL | 0.4 mg/mL |
| Before freeze drying | 2.3% | 2.1% |
| T = 0 | 2.5% | 3.1% |
| 1 month storage of freeze dried vials at | 2.8% | 2.9% |
| +5° C. | | |
| 1 month storage of freeze dried vials at | 2.5% | 3.1% |
| +30° C. | | |
| 1 month storage of freeze dried vials at | 2.3% | 2.8% |
| +40° C. | | |

TABLE 20.3
Oxidized forms %, in formulations of GP-BDD-FVIII with two
different concentrations. The analysis was performed before
freeze drying, after freeze drying (at t = 0), after 1 month and
3 months storage at three different temperatures.

| | oxidised protein % | |
|---|---|---|
| | GP-BDD-FVIII | GP-BDD-FVIII |
| | 0.22 mg/mL | 0.41 mg/mL |
| Before freeze drying | 2.9% | 2.9% |
| T = 0 | 2.8% | 3.0% |
| 1 month storage of freeze dried vials at | 3.0% | 2.9% |
| +5° C. | | |
| 1 month storage of freeze dried vials at | 3.4% | 3.4% |
| +30° C. | | |
| 1 month storage of freeze dried vials at | 3.8% | 3.7% |
| +40° C. | | |
| 3 months storage of freeze dried vials at | 3.0% | 3.2% |
| +5° C. | | |
| 3 months storage of freeze dried vials at | 3.8% | 3.7% |
| +30° C. | | |
| 3 months storage of freeze dried vials at | 4.2% | 4.3% |
| +40° C. | | |

The investigated formulation provides stability of GP-BDD-FVIII at different strengths (protein concentrations). Both investigated strengths of GP-BDD-FVIII were observed to be stable during freeze drying, and after freeze drying. No difference in stability of freeze dried GP-BDD-FVIII is observed when comparing 0.2 mg/mL drug product (corresponding to about 2000 IU/mL) and 0.4 mg/mL drug product (corresponding to about 4000 IU/mL). At 30° C. oxidized forms is increased by about 1 percentage point within three months, and at 40° C. this value is about 1.2-1.3 percentage points.

Additionally, the FVIII activity data shows that GP-BDD-FVIII in freeze dried formulations (suitable for sc administration) is fully active at least during 6 months storage at 30° C.

### Example 21 Sc Formulation with FVIII Glyco-Conjugated with Fab Fragment

In one experiment various FVIII molecules were freeze dried and studied. The investigated FVIII proteins were: GP-BDD-FVIII and Fab-BDD-FVIII (where a Fab-fragment of an antibody was attached at the same position as the PEG polymer of GP-BDD-FVIII and the HEP polymer of GH-BDD-FVIII).

Prior to the freeze drying work, Fab-BDD-FVIII was stored frozen at 1124 U/mL in a buffer containing 3 mg/mL sucrose, 18 mg/mL NaCl, 0.25 mg/mL CaCl₂*2H₂O, 1.5 mg/mL L-histidine, 0.1 mg/mL Tween 80, pH 7.3. GP-BDD-FVIII was stored frozen at 5412 in a buffer containing 12 mg/mL sucrose, 36 mg/mL NaCl, 1 mg/mL CaCl₂*2H₂O, 6 mg/mL L-histidine, 0.4 mg/mL Tween 80, pH 6.9. Both proteins were buffer-exchanged into a buffer containing: 70 mg/mL sucrose, 3.5 mg/mL NaCl, 0.5 mg/mL CaCl₂*2H₂O, 1.55 mg/mL L-histidine, 2.5 mg/mL methionine, 0.4 mg/mL Tween 80. GP-BDD-FVIII was diluted after buffer exchange so that both proteins had strength of 700 IU/mL and a protein concentration about 0.07 mg/mL confirmed by SE-HPLC.

The two formulations were filled into freeze drying vials, with a fill volume of 1 mL. The formulations were freeze dried according to the program described in table 15.1. Both formulations were degassed prior to freeze drying. Degassing took place in the freeze dryer prior to the freezing step according to the procedure described in example 15.

After freeze drying the vials were used for SE-HPLC analysis for quantification of HMWP %.

TABLE 21.1
The percentage of HMWP in reconstituted freeze dried formulations
of GP-BDD-FVIII and Fab-BDD-FVIII

| | HMWP | |
|---|---|---|
| | GP-BDD-FVIII | Fab-BDD-FVIII |
| | 700 IU/mL | 700 IU/mL |
| Before freeze drying | 1.5% | 0.19% |
| T = 0 | 2.2% | 0.26% |

Both protracted FVIII molecules could be formulated in as sc relevant formulation, degassed, freeze dried, and easily reconstituted (1:1 fill volume:reconstitution volume) with only small impact on HMWP %.

### Example 22 Effects of Sucrose

In one experiment freeze dried formulations #3, #4, #5, #6, #7 and #8 contained 2000 IU/mL GP-BDD-FVIII, 3.5 mg/mL NaCl, 2.5 mg/mL methionine, 1.55 mg/mL L-histidine, 0.1 mg/mL polysorbate 20, 1 mg/mL CaCl₂*2H₂O and various amounts of sucrose: 40 mg/mL (#3), 60 mg/mL (#4), 70 mg/mL (#5), 80 mg/mL (#6), 90 mg/mL (#7), 110 mg/mL (#8). A formulation #1 contained 2000 IU/mL GP-BDD-FVIII, 17 mg/mL sucrose, 36 mg/mL NaCl, 0.6 mg/mL methionine, 6 mg/mL L-histidine, 0.4 mg/mL polysorbate 20 and 1 mg/mL CaCl₂*2H₂O, see table 22.1

TABLE 22.1

| | formulation | | | | | | |
|---|---|---|---|---|---|---|---|
| | #1, | #3 | #4 | #5 | #6 | #7 | #8 |
| | N8-GP | | | | | | |
| | 2000 | 2000 | 2000 | 2000 | 2000 | 2000 | 2000 |
| | IU/ | IU/ | IU/ | IU/ | IU/ | IU/ | IU/ |
| | mL | mL | mL | mL | mL | mL | mL |
| Sucrose | 17 | 40 | 60 | 70 | 80 | 90 | 110 |
| NaCl | 36 | 3.5 | 3.5 | 3.5 | 3.5 | 3.5 | 3.5 |
| CaCl2 | 1 | 1 | 1 | 1 | 1 | 1 | 1 |
| (2*H2O) | | | | | | | |
| L-histidine | 6 | 1.55 | 1.55 | 1.55 | 1.55 | 1.55 | 1.55 |
| L-Methionine | 0.6 | 2.5 | 2.5 | 2.5 | 2.5 | 2.5 | 2.5 |
| Tween 20 | 0.4 | 0.1 | 0.1 | 0.1 | 0.1 | 0.1 | 0.1 |
| pH 6.9 | 6.9 | 6.9 | 6.9 | 6.9 | 6.9 | 6.9 | 6.9 |

Formulations were filled into freeze drying vials, with a fill volume of 1 mL. All formulations were freeze dried according to the program described in table 15.1 and one half of the vials were exposed to preceding degassing as described in example 15.

After freeze drying the vials were visually inspected and stored at three different temperatures: −80° C., +30° C. and +40° C., for storage during 3 weeks. It was observed that almost all vials contained nice appearing freeze drying cakes except from freeze drying vials containing formulations #3. Most of the vials containing formulation #3 contained collapsed or partly collapsed freeze drying cakes.

After storage the freeze dried formulations were reconstituted by 1 mL milliQ water and analysed directly by freeze point osmometry to measure the osmolality (table 22.2). Aliquots of reconstituted formulations were stored 3 weeks at −80 C until RP-HPLC analysis for quantification of oxidized protein (oxidized forms %), and for SE-HPLC analyses to quantify HMWP %. These HPLC data is shown in table 22.3, 22.4, 22.5.

TABLE 22.2
The osmolality of seven 2000 IU/mL GP-BDD-FVIII formulations before
freeze drying, and after freeze drying (incl. degassing) and reconstitution.
The content of formulation #1-#8 is described above.

| | Formulation | | | | | | |
|---|---|---|---|---|---|---|---|
| | #1 | #3 | #4 | #5 | #6 | #7 | #8 |
| Osmolality | 1252 | 280 | 345 | 374 | 406 | 438 | 510 |
| (mOsm/kg) | | | | | | | |
| After | | | | | | | |
| reconstitution | | | | | | | |

TABLE 22.3
HMWP % in seven 2000 IU/mL GP-BDD-FVIII formulations.
HWMP % was quantified before freeze drying, after freeze drying
and 3 weeks storage of freeze dried vials at −80° C.,
30° C. and 40° C.

| | HMWP % | | | | | | |
|---|---|---|---|---|---|---|---|
| | #1 | #3 | #4 | #5 | #6 | #7 | #8 |
| Before | 1.6% | 2.1% | 1.9% | 1.9% | 1.9% | 1.9% | 1.9% |
| freeze | | | | | | | |
| drying | | | | | | | |
| 3 weeks | 1.9% | 2.0% | 2.1% | 2.0% | 2.0% | 2.1% | 2.0% |
| at −80° C. | | | | | | | |
| 3 weeks | 2.0% | 2.1% | 2.0% | 2.0% | 2.0% | 2.0% | 2.0% |
| at +30° C. | | | | | | | |
| 3 weeks | 2.0% | 2.0% | 1.9% | 1.9% | 1.8% | 1.8% | 1.7% |
| +40° C. | | | | | | | |

The osmolality of formulations #1 is very high and the formulation is not suitable for sc administration.

The SE-HPLC chromatogram of GP-BDD-FVIII was very similar for all formulations both analysed before and after freeze drying and storage. The largest chromatographic changes associated with freeze drying were observed for F1 (containing high NaCl concentration combined with low sucrose concentration). Highest increase in HMWP % was observed for F1.

The formulation, F3, with 40 mg/mL sucrose was collapsed. Similarities between SE-HPLC chromatograms of F4-F8 formulations indicated that sucrose (60-110 mg/mL) does not affect HMWP % (shown in table 22.3), Monomer % and LMWP % in freeze dried formulations when the investigated formulations contains 1.55 mg/ml L-Histidine, 1 mg/mL CaCl2, 2.5 mg/mL methionine, 3.5 mg/mL NaCl and 0.1 mg/mL tween 20.

TABLE 22.4
Oxidized protein % in seven 2000 IU/mL GP-BDD-FVIII
formulations. Percentage of oxidized protein was quantified
before freeze drying, after freeze drying and 3 weeks storage
at −80° C., 30° C. and 40° C. Formulations were
degassed prior to freeze drying

| | Oxidized protein % | | | | | | |
|---|---|---|---|---|---|---|---|
| | #1 | #3 | #4 | #5 | #6 | #7 | #8 |
| Before | 1.9% | 2.1% | 2.2% | 2.2% | 2.2% | 2.1% | 2.1% |
| freeze | | | | | | | |
| drying | | | | | | | |
| 3 weeks at | 2.3% | 2.4% | 2.5% | 2.5% | 2.4% | 2.4% | 2.6% |
| −80° C. | | | | | | | |
| 3 weeks at | 2.3% | 2.9% | 2.9% | 2.9% | 2.8% | 3.2% | 3.0% |
| +30° C. | | | | | | | |
| 3 weeks | 2.5% | 3.3% | 3.4% | 3.3% | 3.6% | 3.5% | 3.7% |
| +40° C. | | | | | | | |

TABLE 22.5
Oxidized protein % in eight 2000 IU/mL GP-BDD-FVIII
formulations. Percentage of oxidized protein was quantified before
freeze drying, after freeze drying and after storage of freeze dried
vials at 30° C. and 40° C. (1 month). Formulations were
not degassed prior to freeze drying.

| | Oxidized protein % | | | | | | |
|---|---|---|---|---|---|---|---|
| | #1 | #3 | #4 | #5 | #6 | #7 | #8 |
| Before | 1.9% | 2.1% | 2.2% | 2.2% | 2.2% | 2.1% | 2.1% |
| freeze | | | | | | | |
| drying | | | | | | | |
| 3 weeks at | 2.3% | 3.1% | 2.9% | 3.1% | 3.0% | 3.0% | 3.2% |
| −80° C. | | | | | | | |
| 3 weeks at | 2.5% | 5.1% | 4.8% | 4.5% | 4.8% | 5.2% | 5.3% |
| +30° C. | | | | | | | |
| 3 weeks | 2.6% | 6.4% | 6.0% | 6.5% | 7.4% | 7.5% | 7.9% |
| +40° C. | | | | | | | |

The data surprisingly indicates that degassing is only required for formulations where the NaCl concentration was decreased from a high concentration (e.g. 36 mg/mL) to a low concentration (e.g. 3.5 mg/mL). Only minor differences between oxidized forms % is observed for formulation #1 when comparing data in table 22.4 and 22.5.

In contrast comparisons of data in table 22.4 and 22.5 shows significantly effects of degassing for formulations #3-#8 with regards to protein oxidation, as also observed in other studies (example 6 and 7). A large increase in percentage of GP-BDD-FVIII oxidized forms in these formulations is observed after storage at 30° C. and 40° C. when the formulations were not degassed prior to freeze drying.

Surprisingly the content of oxidized forms of FVIII correlates to the sucrose concentration (primarily for formulations stored at 40° C.). The higher the content of sucrose, the higher the content of oxidized forms. This trend is most pronounced for formulations which are not degassed prior to freeze drying and which are stored at 40° C. Sucrose is hereby shown to increase the amount of oxidized FVIII forms in freeze dried formulations. Upon degassing, sucrose can however be used as a stabilizing excipient, allowing low osmolality after reconstitution into small volumes.

### Example 23 in Use Stability/Stability after Reconstitution

This example relates to a previous example (example 22) by investigating the same formulations as described in example 22, yet, with focus on HMWP formation after reconstitution. The freeze dried GP-BDD-FVIII formulations #1, #3-#8 (described in example 22) which had been stored at 40° C. during 3 weeks and which were reconstituted and stored at −80° C. after reconstitution (as described in examples 22) were thawed and placed at 40° C., and then analysed by SE-HPLC for HMWP quantifications after 4 hours at 40° C.

TABLE 23.1
HMWP % in eight 2000 IU/mL GP-BDD-FVIII formulations. HWMP %
was quantified after storage of freeze dried formulations (3 weeks at
40° C.) and after additional storage of reconstituted formulations
(4 hours at 40° C.)

| | HMWP % | | | | | | |
|---|---|---|---|---|---|---|---|
| | #1 | #3 | #4 | #5 | #6 | #7 | #8 |
| 3 weeks +40° C. (also | 2.0% | 2.0% | 1.9% | 1.9% | 1.8% | 1.8% | 1.7% |
| presented in example 22) | | | | | | | |
| 4 hours at 40° C. of | 3.4% | 3.0% | 2.8% | 2.9% | 2.7% | 2.6% | 2.4% |
| reconstituted product | | | | | | | |

The data shows that HMWP is slightly increased during storage (of reconstituted formulations) at 40° C., and that this increase is lower when sucrose concentration is increased. This suggests that the in use stability of a sc GP-BDD-FVIII formulation with high concentration of sucrose is increased compared to the in use stability of a formulations with high NaCl concentration (and high osmolality)

### Example 24 Fill Volume/Reconstitution Volume: 0.3, 0.5 and 0.8 mL

In one experiment freeze dried formulations containing 250, 1500 and 3500 IU/mL GP-BDD-FVIII was prepared with 70 mg/mL sucrose, 3.5 mg/mL NaCl, 2.5 mg/mL methionine, 1.55 mg/mL L-histidine, 0.4 mg/mL polysorbate 20 and 0.5 mg/mL CaCl₂*2H₂O.

Freeze drying vials with the above described formulations were filled with different volumes (fill volume): 0.3 mL, 0.5 mL, 0.8 mL.

All protein solutions were freeze dried according to the program described in table 15.1. All formulations were freeze dried with preceding degassing. The degassing procedure is described in example 15.

After freeze drying all vials were evaluated by visual inspection, and it was confirmed that no cake collapse was observed in any of the vials. All freeze dried formulations were then reconstituted by 10 mM histidine by the same volume as the respective fill volume (0.3, 0.5 or 0.8 mL). The reconstituted formulations were analysed by SE-HPLC to quantify HMWP percentage, data is presented in table 24.1

TABLE 24.1
HMWP % in a formulation of three different strengths
and with three different fill volumes.
HMWP % was quantified before and after freeze drying (t = 0).

| | HMWP % | | |
|---|---|---|---|
| | 250 IU/mL | 1500 IU/mL | 3500 IU/mL |
| Before freeze drying | 1.6% | 2.1% | 2.2% |
| t = 0, Fill vol. 0.3 mL | 1.6% | 2.2% | 2.2% |
| t = 0, Fill vol. 0.5 mL | 1.7% | 2.3% | 2.3% |
| t = 0, Fill vol. 0.8 mL | 1.7% | 2.3% | 2.3% |

The data in table 24.1 shows that there is no effect of fill volume (volume pr vial) on the HMWP % formed during freeze drying.

The data further shows that the HMWP %, before and after freeze, drying is lower in the formulation containing 250 IU/mL (compared to the higher strengths). The HMWP %, is however, similar for 1500 IU/mL and 3500 IU/mL. This is in accordance to the observations in example 20.

### Example 25 Alternative Degassing Procedure

The influence of the degassing procedure on the oxidation of GP-BDD-FVIII was investigated. In this example the GP-BDD-FVIII formulation contains: 2000 IU/mL GP-BDD-FVIII, 3.5 mg/mL NaCl, 0.5 mg/mL CaCl₂, 1.55 mg/mL L-histidine, 2.5 mg/mL methionine, 70 mg/mL sucrose, and 0.4 mg/mL polysorbate 20. Vials with liquid formulation were placed on the shelf of the freeze dryer and the shelf was cooled to 5° C. All formulations were freeze dried according to the program described in table 15. The formulations were degassed in the freeze dryer prior to the freezing step according to following procedure: Oxygen was removed from the liquid by applying low pressure (100 mbar) during 10 minutes at +20° C. The pressure was equilibrated to 1 atm (1013 mbar) by nitrogen gas. The degassing procedure was repeated before the freezing step.

After degassing and freeze drying the freeze dried formulation was reconstituted by 1.1 mL 10 mM histidine, pH 6.0 and analysed by RP-HPLC for oxidized forms (as described in example 3). The percentage of oxidized LC was determined before freeze drying (BFD), right after freeze drying (at T=0), and after storage at 30° C. and 40° C., see data in table 14.2.

TABLE 25.1
Percentage of oxidized protein in freeze dried formulations
of GP-BDD-FVIII degassed for 2 × 10 minutes

| Sample for RP-HPLC analysis | Oxidized forms (%) |
|---|---|
| Before freeze drying | No data |
| After freeze drying | 2.7% |
| T = 0 | |
| 3 weeks storage of freeze dried | 3.1% |
| vials at +30° C. | |
| 3 weeks storage of freeze dried | 3.9% |
| vials at +40° C. | |

Data show that a degassing procedure of 2×10 minutes gives a reduction in the content of oxidised forms after storage for 3 weeks at 30° C. as well as at 40° C., compared to a similar formulation which was not degassed (data in example 7).

1. A freeze dried pharmaceutical FVIII formulation, wherein said formulation comprises a FVIII molecule having an increased in vivo circulatory half-life compared to wt FVIII, wherein said formulation, following reconstitution, is an aqueous isotonic formulation comprising 250-10.000 IU/mL of said FVIII molecule, 1-3 mg NaCl/mL, 0.5-3.0 mg CaCl₂, 2H₂O/mL, and 50-110 mg sucrose/mL.

2. A pharmaceutical formulation according to claim 1, wherein said formulation does not contain any preservatives.

3. A pharmaceutical formulation according to claim 1, wherein said formulation further comprises 0.5-5 mg histidine/mL, 0.5-15 mg methionine/mL, and 0.1-1.0 mg surfactant/mL, and wherein the volume of said reconstituted formulation is 0.3-1.2 mL, and the osmolality is 300-400 mOsm/kg.

4. A pharmaceutical formulation according to claim 1, wherein said FVIII molecule is a B domain truncated molecule having a B domain linker of 15-25 amino acids, wherein said FVIII molecule is conjugated with a half-life extending moiety via an O-glycan linked to the Ser750 amino acid residue according to SEQ ID NO 1, and wherein said FVIII molecule is conjugated with a water soluble polymer selected from PEG and heparosan.

5. A pharmaceutical formulation according to claim 1, wherein said FVIII molecule is a fusion molecule, and wherein the fusion partner of said fusion molecule is selected from the list consisting of: albumin, an Fc domain, and an Fc receptor.

6. A pharmaceutical formulation according to claim 1, wherein said formulation comprises 250-10,000 IU/mL, 3.1 mg histidine/mL, 2.5 mg methionine/mL, 3.0 mg NaCl/mL, 80 mg sucrose/mL, 0.4 mg non-ionic surfactant/mL, and 3 mg CaCl₂, 2H₂O/mL.

7. A pharmaceutical formulation according to claim 1, wherein said formulation comprises 250-10,000 IU FVIII/mL, 3.1 mg histidine/mL, 2.5 mg methionine/mL, 3.5 mg NaCl/mL, 70 mg sucrose/mL, 0.4 mg non-ionic surfactant/mL, and 0.5-2 mg CaCl₂, 2H₂O/mL.

8. A pharmaceutical formulation according to claim 1, wherein said formulation comprises 250-10,000 IU FVIII/mL of said FVIII molecule, 3.1 mg histidine/mL, 2.5 mg methionine/mL, 3.5 mg NaCl/mL, 90 mg sucrose/mL, 0.4 mg non-ionic surfactant/mL, and 2 mg CaCl₂, 2H₂O/mL.

9. A pharmaceutical formulation according to claim 1, wherein said formulation comprises 250-10,000 IU FVIII/mL, 3.1 mg histidine/mL, 2.5 mg methionine/mL, 4 mg NaCl/mL, 80-100 mg sucrose/mL, 0.1-0.4 mg non-ionic surfactant/mL, and 2 mg CaCl₂, 2H₂O/mL.

10. A pharmaceutical formulation according to claim 1, wherein said formulation comprises 250-10,000 IU FVIII/mL, 3.1 mg histidine/mL, 2.5 mg methionine/mL, 4-6 mg NaCl/mL, 100 mg sucrose/mL, 0.1-0.4 mg non-ionic surfactant/mL, and 2 mg CaCl₂, 2H₂O/mL.

11. A pharmaceutical formulation according to claim 1, wherein said formulation comprises 250-10,000 IU FVIII/mL, 3.1 mg histidine/mL, 2.5 mg methionine/mL, 4 mg NaCl/mL, 70-90 mg sucrose/mL, 0.1-0.4 mg non-ionic surfactant/mL, and 1-5 mg CaCl₂, 2H₂O/mL.

12. A pharmaceutical formulation according to claim 1, wherein said formulation comprises 250-10,000 IU FVIII/mL, 1-4 mg histidine/mL, 2.5 mg methionine/mL, 7 mg NaCl/mL, 100 mg sucrose/mL, 0.4 mg non-ionic surfactant/mL, and 2 mg CaCl₂, 2H₂O/mL.

13. A pharmaceutical formulation according to claim 1, wherein said formulation comprises 250-10,000 IU FVIII/mL, 1.5 mg/ml histidine, 2.5 mg methionine/mL, 3.5 mg NaCl/mL, 70 mg sucrose/mL, 0.4 mg non-ionic surfactant/mL, and 0.5-1 mg CaCl₂, 2H₂O/mL.

14. A pharmaceutical formulation according to claim 1, wherein said formulation comprises 250-10,000 IU FVIII/mL, 2-4 mg histidine/mL, 2.5 mg methionine/mL, 1.5 mg NaCl/mL, 70 mg sucrose/mL, 0.4 mg non-ionic surfactant/mL, and 1.5 mg CaCl₂, 2H₂O/mL.

15. A pharmaceutical formulation according to claim 1, wherein the amount of oxidized FVIII light chain molecules is below 5% of the total amount of FVIII following storage of the formulation at 30° C. for three months.

16. A pharmaceutical formulation according to claim 1, wherein the formulation is reconstituted in 10 mM histidine solution, and wherein the volume of the reconstituted formulation is up to 1 mL.

17. A pharmaceutical formulation according to claim 1, wherein the freeze dried formulation, prior to reconstitution, is a pharmaceutically elegant freeze dried cake with a volume that essentially corresponds to the fill volume before freeze drying.

18. A freeze dried pharmaceutical FVIII formulation according to claim 1, wherein the amount of oxidized FVIII light chain molecules is below 5% of the total amount of FVIII, following storage at 30° C. for 3 months.

19. A process for making a freeze dried pharmaceutical formulation according to claim 1, wherein said process comprises the steps of (i) degassing the liquid formulation under low pressure, (ii) pressure equilibrating the degassed formulation with an inert gas, and (iii) freeze drying the degassed formulation.

20. A pharmaceutical formulation obtained by the method according to claim 19.

21. A pharmaceutical formulation according to claim 1, wherein said formulation is for subcutaneous administration.

22. A pharmaceutical formulation according to claim 1, wherein said formulation is for intravenous administration.

23. A pharmaceutical formulation according to claim 1 for use in treatment of haemophilia A.


SEQUENCE LISTING (10 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 21 aa, Artificial Sequence
  Synthetic
SFSQNSRHPSQNPPVLKRHQR

SEQ ID NO: 3 - protein, 20 aa, Artificial Sequence
  Synthetic
SFSQNSRHPSQNPPVLKRHQ

SEQ ID NO: 4 - protein, 20 aa, Artificial Sequence
  Synthetic
FSQNSRHPSQNPPVLKRHQR

SEQ ID NO: 5 - protein, 226 aa, Artificial Sequence
  Synthetic
SFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSS

SEQ ID NO: 6 - protein, 585 aa, Artificial Sequence [sequence omitted]

SEQ ID NO: 7 - protein, 273 aa, Artificial Sequence
  Synthetic
QVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTP

SEQ ID NO: 8 - protein, 227 aa, Artificial Sequence
  Synthetic
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 28 aa, Artificial Sequence
  Synthetic
SSSSKAPPPSLPSPSRLPGPSDTPILPQ

SEQ ID NO: 10 - protein, 865 aa, Artificial Sequence
  Synthetic
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPG